(19) United States
(12) Patent Application Publication
(10) Pub. No.: US 2026/0083598 A1
(43) Pub. Date: Mar. 26, 2026

(54) FLEXIBLE ABSORBENT ARTICLE

(71) Applicant: Kimberly-Clark Worldwide, Inc., Neenah, WI (US)

(72) Inventors: Hwa Y. Kim, Greenville, WI (US); Lori A. Roocks, Menasha, WI (US); Matthew R. Van Hout, Appleton, WI (US); Adam J. Kurzynski, Neenah, WI (US)

(21) Appl. No.: 19/109,836

(22) PCT Filed: Sep. 29, 2023
(86) PCT No.: PCT/US23/34073

§ 371 Date: Mar. 7, 2025

Related U.S. Application Data
(60) Provisional application No. 63/412,093, filed on Sep. 30, 2022.

(51) Int. Cl. A61F 13/49; A61F 13/494

(52) U.S. Cl. CPC A61F 13/49017; A61F 13/49019; A61F 13/49406

(57) ABSTRACT

The present disclosure is directed toward absorbent articles having increased stiffness. In particular, an absorbent article has a body side liner, an outer cover, and an absorbent system comprising a fluid acquisition layer disposed between the bodyside liner and the outer cover. The article further comprises elasticated containment flaps having an active region and one or more inactive regions. The article has an article flexibility of less than or equal to 35 gf at a first location of the article located within the containment flap first inactive regions, and wherein the absorbent article has an article flexibility of greater than or equal to 40 gf at a second location of the article located within the containment flap active region, the article flexibility determined according to the Probe Flexibility Test Method described herein.

### TECHNICAL FIELD

The present disclosure is directed to absorbent articles and more specifically to absorbent articles with high flexibility.

### BACKGROUND OF THE DISCLOSURE

A primary function of personal care absorbent articles is to absorb and retain body exudates such as urine, fecal material, blood, and menses with additional desired attributes including low leakage of the exudates from the absorbent article and a dry feel to the wearer of the absorbent article. By preventing leakage of the exudates from the absorbent article, the absorbent article intends to prevent the body exudates from soiling or contaminating a wearer's or caregiver's clothing or other articles, such as bedding, that can come in contact with the wearer.

As such absorbent articles are worn by users, fit and comfort of such absorbent articles are important considerations for a successful product. One area of exploration to improve fit and comfort of such absorbent articles is through product thinness. In some instances, thinner products may be seen as fitting better and being more comfortable. However, thin products introduce new challenges to overall article construction which can affect the fit and comfort of such articles as well as other features such as product appearance. Advances in absorbent article construction to accommodate thin constructions are continually desirable.

### SUMMARY OF THE DISCLOSURE

According to a first embodiment, an absorbent article has a central longitudinal axis and has a longitudinal length extending between a front waist edge and a rear waist edge and extends in a lateral direction between longitudinally extending side edges, the absorbent article comprises: a front waist region, a rear waist region, and a crotch region disposed between the front waist region and the rear waist region, a bodyside liner, an outer cover, an absorbent system disposed between the bodyside liner and the outer cover, the absorbent system comprising one or more absorbent layers, a fluid acquisition layer, a first longitudinally extending containment flap comprising a barrier material and one or more longitudinally extending elastic members disposed on a first side of the central longitudinal axis, and a second longitudinally extending containment flap comprising a barrier material and one or more longitudinally extending elastic members disposed on a second side of the central longitudinal axis, each of the pair of longitudinally extending containment flaps having an active region and one or more inactive regions, and leg elastic members disposed outboard of each of the first and second longitudinally extending containment flaps and forming an active leg elastic region between the front waist edge and the rear waist edge, wherein each of the pair of longitudinally extending containment flaps have first inactive regions extending from the front waist edge toward the rear waist edge, the first inactive regions extend for at least 10% of the article longitudinal length, and wherein each of the pair of longitudinally extending containment flaps have an active region disposed between the first inactive regions and the rear waist edge, wherein the absorbent article has an article flexibility of less than or equal to 35 gf at a first location of the article located within the containment flap first inactive regions, and wherein the absorbent article has an article flexibility of greater than or equal to 40 gf at a second location of the article located within the containment flap active region, the article flexibility determined according to the Probe Flexibility Test Method described herein.

According to a second embodiment, an absorbent article has a central longitudinal axis and has a longitudinal length extending between a front waist edge and a rear waist edge and extends in a lateral direction between longitudinally extending side edges, the absorbent article comprises a front waist region, a rear waist region, and a crotch region disposed between the front waist region and the rear waist region, a bodyside liner, an outer cover, an absorbent system disposed between the bodyside liner and the outer cover, the absorbent system comprising one or more absorbent layers and further comprising a rear region, a middle region, and a front region, the absorbent system comprised of absorbent material with the absorbent material disposed at a basis weight throughout the absorbent system, wherein an absorbent material basis weight of any one of the rear region, of the middle region, and of the front region does not vary by more than 20% relative to an absorbent material basis weight of any other one of the rear region, the middle region, and the front region, a fluid acquisition layer extending between a front edge disposed proximate the article front waist edge and a rear edge disposed proximate the article rear waist edge, a first longitudinally extending containment flap comprising a barrier material and one or more longitudinally extending elastic members disposed on a first side of the central longitudinal axis, and a second longitudinally extending containment flap comprising a barrier material and one or more longitudinally extending elastic members disposed on a second side of the central longitudinal axis, each of the pair of longitudinally extending containment flaps having an active region and one or more inactive regions, and leg elastic members disposed outboard of each of the first and second longitudinally extending containment flaps and forming active leg elastic region between the front waist edge and the rear waist edge, wherein each of the pair of longitudinally extending containment flaps have first inactive regions extending from the front waist edge toward the rear waist edge, the first inactive regions extend for at least 10% of the article longitudinal length, and wherein each of the pair of longitudinally extending containment flaps have active regions disposed between the first inactive regions and the rear waist edge, wherein the fluid acquisition layer front edge is disposed no further away from the absorbent article front waist edge than the active regions of each of the pair of longitudinally extending containment flaps, and wherein the absorbent article has an article flexibility of less than or equal to 40 gf at a first location of the article located within the first inactive regions and where the absorbent system is present, the article flexibility determined according to the Probe Flexibility Test Method described herein.

In a third embodiment, an absorbent article has a central longitudinal axis and has a longitudinal length extending between a front waist edge and a rear waist edge and extends in a lateral direction between longitudinally extending side edges, the absorbent article comprises a front waist region, a rear waist region, and a crotch region disposed between the front waist region and the rear waist region, a bodyside liner, an outer cover, an absorbent system extending between an absorbent system front edge disposed proximate the article front waist edge and an absorbent system rear edge disposed proximate the article rear waist edge and disposed between the bodyside liner and the outer cover, the absorbent system comprising one or more absorbent layers, a fluid acquisition layer extending between a fluid acquisition layer front edge disposed proximate the article front waist edge and a fluid acquisition layer rear edge disposed proximate the article rear waist edge, wherein the fluid acquisition layer front edge is disposed closer to the article rear waist edge than the absorbent system front edge, a first longitudinally extending containment flap comprising a barrier material and one or more longitudinally extending elastic members disposed on a first side of the central longitudinal axis, and a second longitudinally extending containment flap comprising a barrier material and one or more longitudinally extending elastic members disposed on a second side of the central longitudinal axis, each of the pair of longitudinally extending containment flaps having an active region and one or more inactive regions, and leg elastic members disposed outboard of each of the first and second longitudinally extending containment flaps and forming active leg elastic region between the front waist edge and the rear waist edge, wherein each of the pair of longitudinally extending containment flaps have first inactive regions extending from the front waist edge toward the rear waist edge, the first inactive regions extend for at least 10% of the article longitudinal length, and wherein each of the pair of longitudinally extending containment flaps have active regions disposed between the first inactive regions and the rear waist edge, wherein the fluid acquisition layer front edge is disposed no further away from the absorbent article front waist edge than the active regions of each of the pair of longitudinally extending containment flaps, and wherein the absorbent article has an article flexibility of less than or equal to 40 gf at a first location of the article located within the first inactive regions and where the absorbent system is present, the article flexibility determined according to the Probe Flexibility Test Method described herein.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is side perspective view of an exemplary embodiment of an absorbent article, such as a diaper, in a fastened condition.

FIG. 2A is a top plan view of the absorbent article of FIG. 1 in a stretched, laid flat, unfastened condition as viewed from the body facing surface.

FIG. 2B is a top plan view of the absorbent article of FIG. 1 in a stretched, laid flat, unfastened condition as viewed from the outer cover facing surface.

FIG. 3 is a front perspective view of an alternative embodiment of an absorbent article, such as a pant.

FIG. 4 is a top plan view of the absorbent article of FIG. 3 in a stretched, laid flat condition.

FIG. 5 is a front perspective cross-sectional view taken along line 5-5 from FIG. 2, with the absorbent article being in a relaxed configuration.

Repeat use of reference characters in the present specification and drawings is intended to represent the same or analogous features or elements of the disclosure.

### DETAILED DESCRIPTION OF THE DISCLOSURE

In an embodiment, the present disclosure is generally directed towards absorbent cores absorbent material comprising a high proportion of superabsorbent material. Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products and other adult care garments, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

“Elastomeric” refers to a material or composite which can be elongated by at least 50 percent of its relaxed length and which will recover, upon release of the applied force, at least 20 percent of its elongation. It is generally preferred that the elastomeric material or composite be capable of being elongated by at least 50 percent, more preferably by at least 100 percent, and still more preferably by at least 300 percent of its relaxed length and recover, upon release of an applied force, at least 50 percent of its elongation.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, educative drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than 0.3, and in an embodiment, between 0.6, 5 and 10 and 15, 20 and 40. Spunbond fibers are generally not tacky when they are deposited on a collecting surface.

The term “superabsorbent” refers herein to a water-swellable, water-insoluble organic or inorganic material capable, under the most favorable conditions, of absorbing at least 15 times its weight and, in an embodiment, at least 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “super-majority” refers herein to a majority of at least 65%.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

### Absorbent Article:

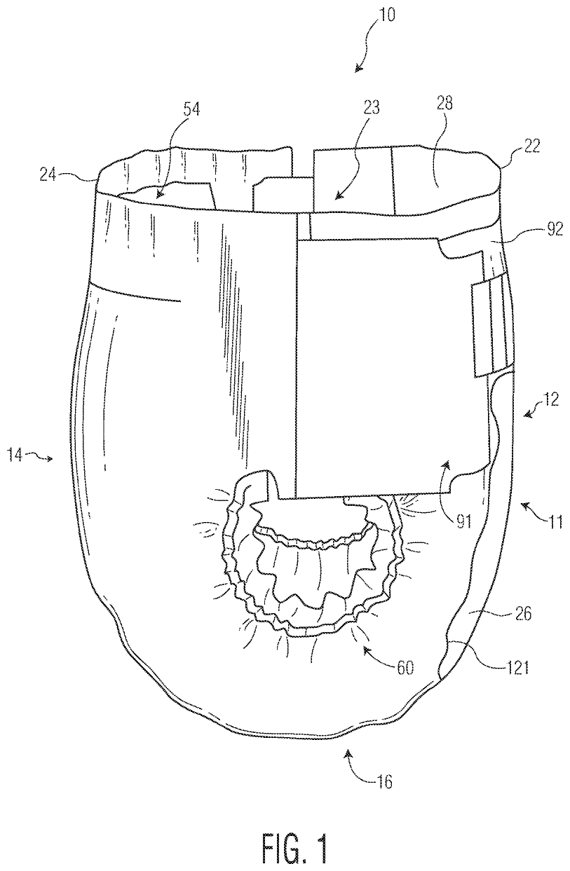

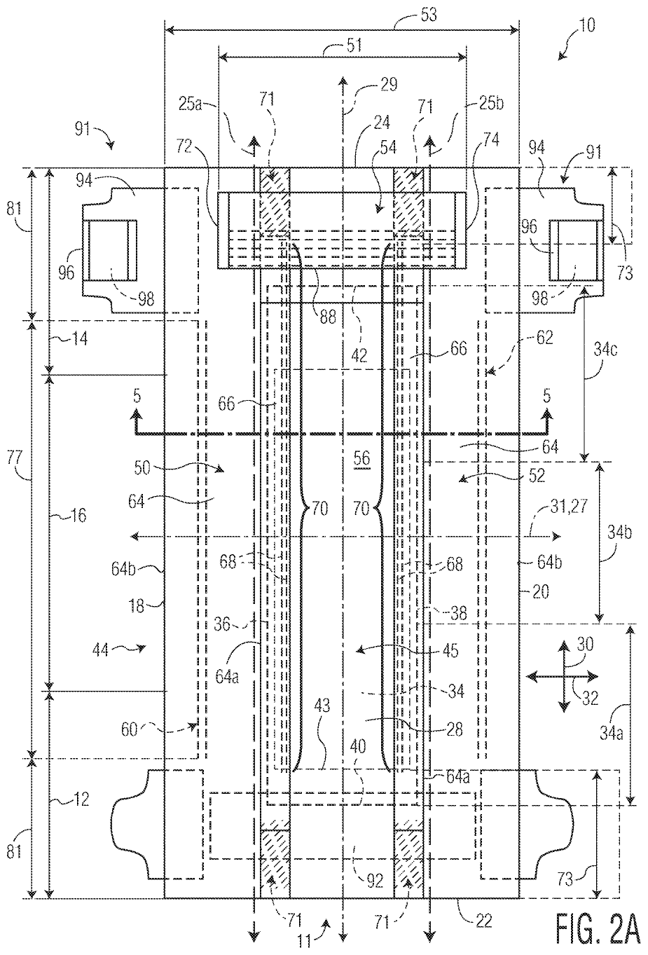

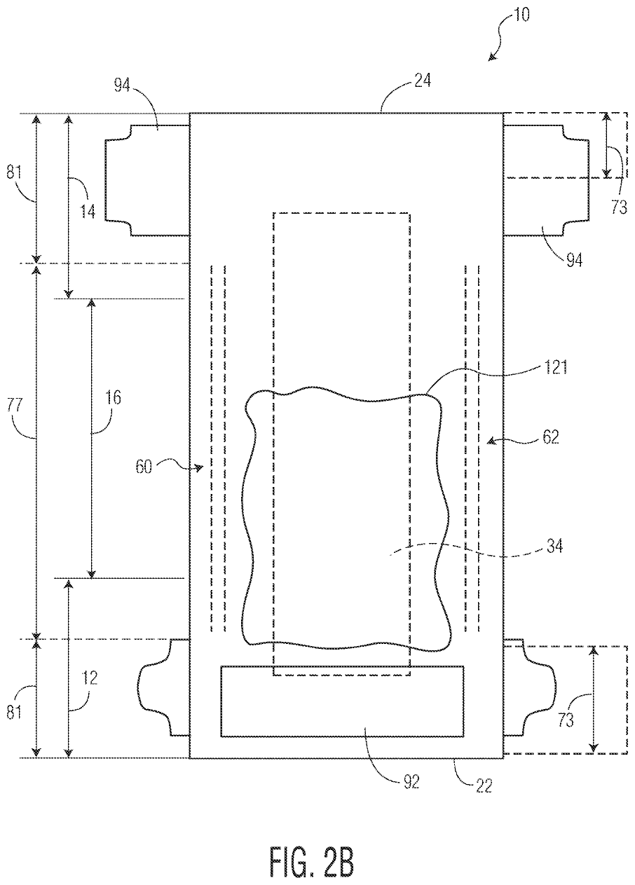

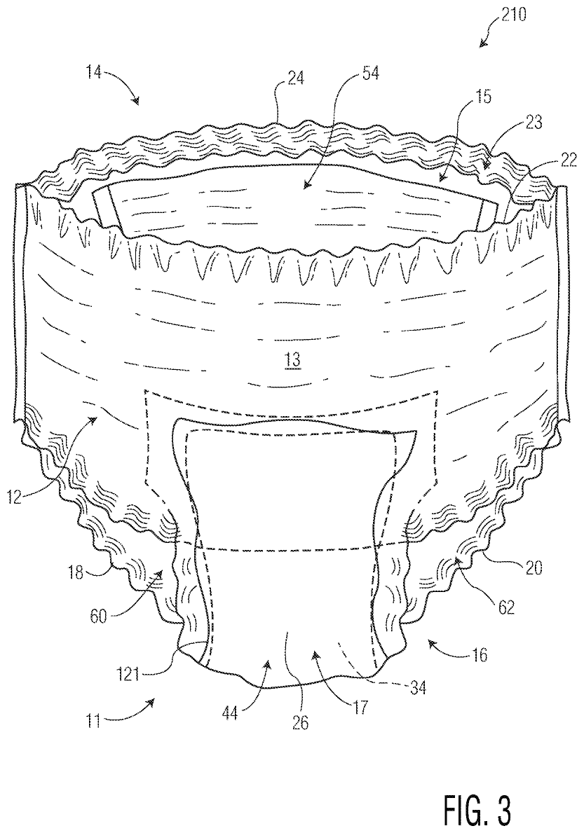

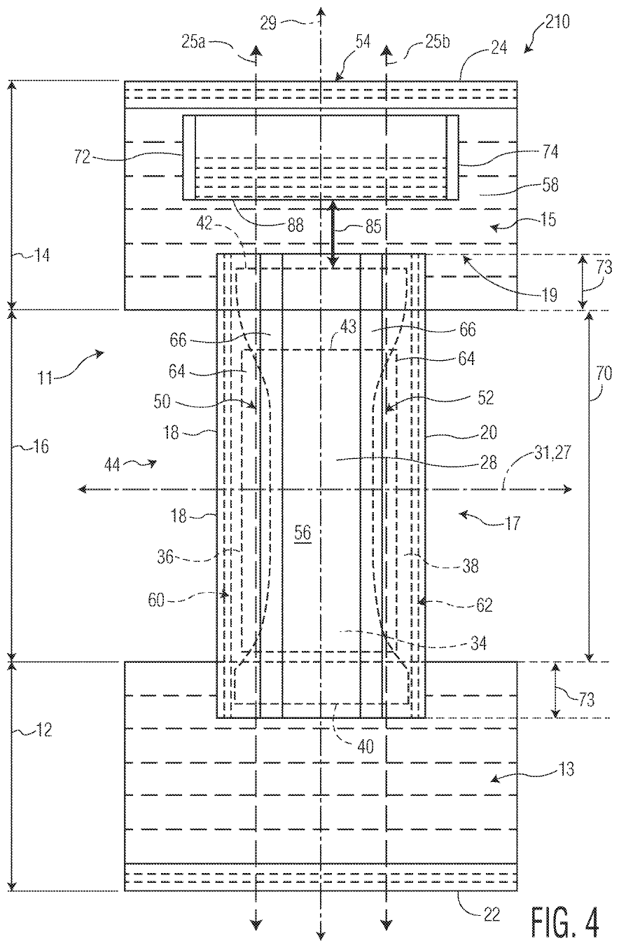

Referring to FIGS. 1-2, a non-limiting illustration of an absorbent article 10, for example a diaper, is illustrated. While the embodiments and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross-machine direction manufacturing of a product, without departing from the spirit and scope of the disclosure. For example, the absorbent article 210 in FIGS. 3-4 provide an exemplary embodiment of an absorbent article 210 that can be manufactured in cross-machine direction manufacturing process.

As will be described in more detail below, such articles 10, 210 of the present disclosure can be particularly thin and flexible. For example, particular absorbent systems of such articles 10, 210 comprising very low amounts of pulp fluff allow such absorbent systems to be very thin and flexible and thus result in very thin and flexible articles 10, 210. The thin and flexibleness of such articles 10, 210 are beneficial to wearers from a fit and comfort perspective. However, the thinness and flexibility also introduce new challenges to such articles 10, 210. In some thin and flexible articles 10, 210 the elastic tensions within the articles provide such retractive forces that the articles 10, 210 form creases or folds in a relaxed configuration. These creases or folds can interfere with the look of the articles 10, 210 and/or distort graphical elements of the 10, 210 in an undesirable manner.

Turning to the construction of the articles of the present disclosure, absorbent article 10 illustrated in FIGS. 1 and 2 and the absorbent article 210 illustrated in FIGS. 3 and 4 can each include a chassis 11. The absorbent article 10, 210 can include a front waist region 12, a rear waist region 14, and a crotch region 16 disposed between the front waist region 12 and the rear waist region 14 and interconnecting the front and rear waist regions, 12, 14, respectively. The front waist region 12 can be referred to as the front-end region, the rear waist region 14 can be referred to as the rear-end region, and the crotch region 16 can be referred to as the intermediate region. In the embodiment depicted in FIGS. 3 and 4, a three-piece construction of an absorbent article 210 is depicted where the absorbent article 210 can have a chassis 11 including a front waist panel 13 defining the front waist region 12, a rear waist panel 15 defining the rear waist region 14, and an absorbent panel 17 defining the crotch region 16 of the absorbent article 210. The absorbent panel 17 can extend between the front waist panel 13 and the rear waist panel 15. In some embodiments, the absorbent panel 17 can overlap the front waist panel 13 and the rear waist panel 15. The absorbent panel 17 can be bonded to the front waist panel 13 and the rear waist panel 15 to define a three-piece construction. However, it is contemplated that an absorbent article can be manufactured in a cross-machine direction without being a three-piece construction garment.

The absorbent article 10, 210 can have a pair of longitudinal side edges 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24. The longitudinal side edges 18, 20 can extend in a direction parallel to the longitudinal direction 30 for their entire length, such as for the absorbent article 10 illustrated in FIGS. 1 and 2. In other embodiments, the longitudinal side edges 18, 20 can be curved between the front waist edge 22 and the rear waist edge 24. In the absorbent article 210 of FIGS. 3 and 4, the longitudinal side edges 18, 20 can include portions of the front waist panel 13, the absorbent panel 17, and the rear waist panel 15.

The front waist region 12 can include the portion of the absorbent article 10, 210 that, when worn, is positioned at least in part on the front of the wearer while the rear waist region 14 can include the portion of the absorbent article 10, 210 that, when worn, is positioned at least in part on the back of the wearer. The crotch region 16 of the absorbent article 10, 210 can include the portion of the absorbent article 10, 210 that, when worn, is positioned between the legs of the wearer and can partially cover the lower torso of the wearer. The waist edges, 22 and 24, of the absorbent article 10, 210 are configured to encircle the waist of the wearer and together define a central waist opening 23 (as labeled in FIG. 1 and FIG. 3) for the waist of the wearer. Portions of the longitudinal side edges 18, 20 in the crotch region 16 can generally define leg openings for the legs of the wearer when the absorbent article 10, 210 is worn.

The absorbent article 10, 210 can include an outer cover 26 and a bodyside liner 28. The outer cover 26 and the bodyside liner 28 can form a portion of the chassis 11. In an embodiment, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated embodiment, can coincide with the length and width of the absorbent article 10. As illustrated in FIGS. 2 and 4, the absorbent article 10, 210 can have a longitudinal axis 29 extending in the longitudinal direction 30 and a lateral axis 31 extending in the lateral direction 32.

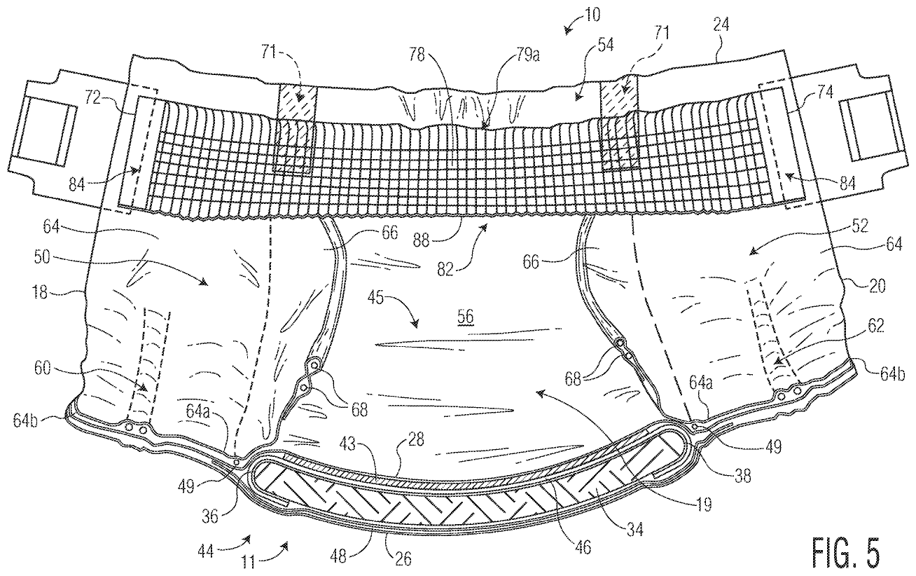

The chassis 11 can include an absorbent system 34. The absorbent system 34 can be disposed between the outer cover 26 and the bodyside liner 28. The absorbent system 34 can have longitudinal edges, 36 and 38, which, in an embodiment, can form portions of the longitudinal side edges, 18 and 20, respectively, of the absorbent article 10, 210. The absorbent system 34 can have a first end edge 40 that is opposite a second end edge 42, respectively, which, in an embodiment, can form portions of the waist edges, 22 and 24, respectively, of the absorbent article 10. In some embodiments, the first end edge 40 can be in the front waist region 12. In some embodiments, the second end edge 42 can be in the rear waist region 14. In an embodiment, the absorbent system 34 can have a length and width that are the same as or less than the length and width of the absorbent article 10, 210. The bodyside liner 28, the outer cover 26, and the absorbent system 34 can form part of an absorbent assembly 44. In the absorbent article 210 of FIGS. 3 and 4, the absorbent panel 17 can form the absorbent assembly 44. The absorbent assembly 44 can also include a fluid transfer layer 46 (as shown in FIG. 5) and a fluid acquisition layer 43 between the bodyside liner 28 and the fluid transfer layer 46 as is known in the art. Such a fluid transfer layer 46 is sometimes alternatively known as a corewrap. The fluid acquisition layer 43 is sometime alternatively known as a surge layer. The absorbent assembly 44 can also include a spacer layer 48 (as shown in FIG. 5) disposed between the absorbent system 34 and the outer cover 26. Still further embodiments of the absorbent assembly 44 are contemplated. For example, the absorbent assembly 44 may include a further corewrap material disposed opposite the fluid transfer layer 46 and between the absorbent system 34 and the space layer 48. The spacer layer 48 may be present in some of these embodiments and absent in other embodiments.

The absorbent article 10, 210 can be configured to contain and/or absorb liquid, solid, and semi-solid body exudates discharged from the wearer. In some embodiments, containment flaps 50, 52 can be configured to provide a barrier to the lateral flow of body exudates. To further enhance containment and/or absorption of body exudates, the absorbent article 10, 210 can suitably include a waist containment member 54. In some embodiments, the waist containment member 54 can be disposed in the rear waist region 14 of the absorbent article 10, 210. Although not depicted herein, it is contemplated that the waist containment member 54 can be additionally or alternatively disposed in the front waist region 12 of the absorbent article 10, 210.

The waist containment member 54 can be disposed on the body facing surface 19 of the chassis 11 to help contain and/or absorb body exudates. In some embodiments, such as in the absorbent articles 10 depicted in FIGS. 1 and 2, the waist containment member 54 can be disposed on the body facing surface 45 of the absorbent assembly 44. In some embodiments, the waist containment member 54 can be disposed on the body facing surface 56 of the bodyside liner 28. In some embodiments, such as in the absorbent article 210 depicted in FIGS. 3 and 4, the waist containment member 54 can be disposed on the body facing surface 58 of the rear waist panel 15.

The absorbent article 10, 210 can further include leg elastic members 60, 62 as are known to those skilled in the art. The leg elastic members 60, 62 can be attached to the outer cover 26 and/or the bodyside liner 28 along the opposite longitudinal side edges, 18 and 20, and positioned in the crotch region 16 of the absorbent article 10, 210. The leg elastic members 60, 62 can be parallel to the longitudinal axis 29 as shown in FIGS. 2 and 4 or can be curved as is known in the art. The leg elastic members 60, 62 can be elastomeric and can provide elasticized leg cuffs.

In some embodiments, the absorbent article 10, 210 can further include longitudinal extending fold lines 25a, 25b, as shown in FIGS. 2 and 4. The first longitudinal extending fold line 25a can be on one side of the longitudinal axis 29 of the absorbent article 10, 210 and the second longitudinal extending fold line 25b can be on an opposite side of the longitudinal axis 29. In some embodiments, the longitudinal extending fold lines 25a, 25b can be generally parallel to the longitudinal axis 29 of the absorbent article 10, 210. In some embodiments, the absorbent article 10, 210 can further include a lateral extending fold line 27. The lateral extending fold line 27 can be parallel to and located at the lateral axis 31 of the absorbent article 10, 210 in some embodiments.

Additional details regarding each of these elements of the absorbent article 10, 210 described herein can be found below and with reference to the Figures.

### Outer Cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10, 210. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In some embodiments, the outer cover 26 can be a two-layer construction, including an outer layer (not shown) and an inner layer (not shown) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer of the outer cover 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer of an outer cover 26 can be a 20 gsm spunbond polypropylene non-woven web. The outer layer may also be constructed of the same materials from which the bodyside liner 28 can be constructed as described herein.

The liquid impermeable inner layer of the outer cover 26 (or the liquid impermeable outer cover 26 where the outer cover 26 is of a single-layer construction) can be either vapor permeable (i.e., “breathable”) or vapor impermeable. The liquid impermeable inner layer (or the liquid impermeable outer cover 26 where the outer cover 26 is of a single-layer construction) can be manufactured from a thin plastic film. The liquid impermeable inner layer (or the liquid impermeable outer cover 26 where the outer cover 26 is of a single-layer construction) can inhibit liquid body exudates from leaking out of the absorbent article 10, 210 and wetting articles, such as bed sheets and clothing, as well as the wearer and caregiver.

In some embodiments, where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

### Bodyside Liner:

The bodyside liner 28 of the absorbent article 10, 110, 210 can overlay the absorbent system 34 and the outer cover 26 and can isolate the wearer's skin from liquid waste retained by the absorbent system 34. In various embodiments, a fluid transfer layer 46 can be positioned between the bodyside liner 28 and the absorbent system 34. In various embodiments, an acquisition layer (not shown) can be positioned between the bodyside liner 28 and the absorbent system 34 or a fluid transfer layer 46, if present. In various embodiments, the bodyside liner 28 can be bonded to the acquisition layer, or to the fluid transfer layer 46 if no acquisition layer is present, via adhesive and/or by a point fusion bonding. The point fusion bonding may be selected from ultrasonic, thermal, pressure bonding, and combinations thereof.

In an embodiment, the bodyside liner 28 can extend beyond the absorbent system 34 and/or a fluid transfer layer 46, if present, and/or an acquisition layer, if present, and/or a spacer layer 48, if present, to overlay a portion of the outer cover 26 and can be bonded thereto by any method deemed suitable, such as, for example, by being bonded thereto by adhesive, to substantially enclose the absorbent system 34 between the outer cover 26 and the bodyside liner 28. The bodyside liner 28 may be narrower than the outer cover 26. However, in other embodiments, the bodyside liner 28 and the outer cover 26 may be of the same dimensions in width and length. In other embodiments, the bodyside liner 28 can be of greater width than the outer cover 26. It is also contemplated that the bodyside liner 28 may not extend beyond the absorbent system 34 and/or may not be secured to the outer cover 26. In some embodiments, the bodyside liner 28 can wrap at least a portion of the absorbent system 34, including wrapping around both longitudinal edges 36, 38 of the absorbent system 34, and/or one or more of the end edges 40, 42. It is further contemplated that the bodyside liner 28 may be composed of more than one segment of material. The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent system 34 to permit body exudates to readily penetrate through to the absorbent system 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Pat. No. 9,474,660 to Kirby, Scott S. C. et al., the entirety of which is hereby incorporated by reference.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from 10 or 12 to 15 or 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral or circumferential direction of the absorbent article 10, 210. In other aspects, the bodyside liner 28 can be stretchable, and more suitably elastic, in both the lateral and the longitudinal directions 32, 30, respectively.

### Containment Flaps:

In an embodiment, the absorbent article 10, 210 can include a pair of containment flaps 50, 52. The containment flaps 50, 52 can be formed separately from the absorbent chassis 11 and attached to the chassis 11 or can be formed integral to the chassis 11. In some embodiments, the containment flaps 50, 52 can be secured to the chassis 11 of the absorbent article 10, 210 in a generally parallel, spaced relation with each other laterally inward of the leg openings to provide a barrier against the flow of body exudates. One containment flap 50 can be on a first side of the longitudinal axis 29 and the other containment flap 52 can be on a second side of the longitudinal axis 29. In an embodiment, the containment flaps 50, 52 can extend generally in a longitudinal direction 30 from the front waist region 12 of the absorbent article 10, through the crotch region 16 to the rear waist region 14 of the absorbent article 10. In some embodiments, the containment flaps 50, 52 can extend in a direction substantially parallel to the longitudinal axis 29 of the absorbent article 10, 210, however, in other embodiments, the containment flaps 50, 52 can be curved, as is known in the art. In other embodiments, such as the absorbent article 210 in FIGS. 3 and 4, the containment flaps 50, 52 can be disposed on the absorbent panel 17 in the crotch region 16.

In embodiments where the containment flaps 50, 52 are coupled to the chassis 11, the containment flaps 50, 52 can be bonded to the bodyside liner 28 with a barrier adhesive 49, as shown in FIG. 5. Alternatively, the containment flaps 50, 52 can be bonded to the outer cover 26 with a barrier adhesive 49, or to the spacer layer 48. Of course, the containment flaps 50, 52 can be bonded to other components of the chassis 11 and can be bonded with other suitable means other than a barrier adhesive 49. The containment flaps 50, 52 can be constructed of a fibrous material which can be similar to the material forming the bodyside liner 28. Other conventional materials, such as polymer films, can also be employed.

The containment flaps 50, 52 can each include a base portion 64 and a projection portion 66. The base portion 64 can be bonded to the chassis 11, for example, to the bodyside liner 28 or the outer cover 26 as mentioned above. The base portion 64 can include a proximal end 64a and a distal end 64b. The projection portion 66 can be separated from the base portion 64 at the proximal end 64a of the base portion 64. As used in this context, the projection portion 66 is separated from the base portion 64 at the proximal end 64a of the base portion 64 in that the proximal end 64a of the base portion 64 defines a transition between the projection portion 66 and the base portion 64. The proximal end 64a of the base portion 64 can be located near the barrier adhesive 49. In some embodiments, the distal ends 64b of the base portion 64 can laterally extend to the respective longitudinal side edges 18, 20 of the absorbent article 10, 210. In other embodiments, the distal ends 64b of the base portion 64 can end laterally inward of the respective longitudinal side edges 18, 20 of the absorbent article 10, 210. The containment flaps 50, 52 can also each include a projection portion 66 that is configured to extend away from the body facing surface 19 of the chassis 11 at least in the crotch region 16 when the absorbent article 10, 210 is in a relaxed configuration, as illustrated in FIG. 5. The containment flaps 50, 52 can include a tack-down region 71 in either or both of the front waist region 12 and the rear waist region 14 where the projection portion 66 is coupled to the body facing surface 19 of the chassis 11.

It is contemplated that the containment flaps 50, 52 can be of various configurations and shapes, and can be constructed by various methods. For example, the containment flaps 50, 52 of FIG. 5 depict a vertical containment flap 50, 52 with a tack-down region 71 in both the front and rear waist regions 12, 14 where the projection portion 66 of each containment flap 50, 52 is tacked down to the bodyside liner 28 towards or away from the longitudinal axis 29 of the absorbent article 10, 210. However, the containment flaps 50, 52 can include a tack-down region 71 where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 could be constructed in a “T-shape” configuration, such as described in U.S. Pat. No. 9,259,362 by Robert L. Popp et al. Such a configuration can also include a tack-down region 71 in either or both of the front and rear waist regions 12, 14, respectively. Of course, other configurations of containment flaps 50, 52 can be used in the absorbent article 10, 210 and still remain within the scope of this disclosure.

The containment flaps 50, 52 can include one or more flap elastic members 68, such as the two flap elastic strands depicted in FIG. 5. Suitable elastomeric materials for the flap elastic members 68 can include sheets, strands or ribbons of natural rubber, synthetic rubber, or thermoplastic elastomeric materials. Of course, while two elastic members 68 are shown in each containment flap 50, 52, it is contemplated that the containment flaps 50, 52 can be configured with one or three or more elastic members 68. Alternatively or additionally, the containment flaps 50, 52 can be composed of a material exhibiting elastomeric properties itself.

The flap elastic members 68, as illustrated in FIG. 5, can have two strands of elastomeric material extending longitudinally in the projection portion 66 of the containment flaps 50, 52, in generally parallel, spaced relation with each other. The elastic members 68 can be within the containment flaps 50, 52 while in an elastically contractible condition such that contraction of the strands gathers and shortens the projection portions 66 of the containment flaps 50, 52 in the longitudinal direction 30. As a result, the elastic members 68 can bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, 210, when the absorbent article 10 is in a relaxed configuration.

During manufacture of the containment flaps 50, 52 at least a portion of the elastic members 68 can be bonded to the containment flaps 50, 52 while the elastic members 68 are elongated. The percent elongation of the elastic members 68 can be, for example, 110% to 350%. In one embodiment, the elastic members 68 can be coated with adhesive while elongated to a specified length prior to attaching to the elastic members 68 to the containment flaps 50, 52. In a stretched condition, the length of the elastic members 68 which have adhesive coupled thereto can provide an active flap elastic region 70 in the containment flaps 50, 52, as labeled in FIG. 2, which will gather upon relaxation of the absorbent article 10. The active flap elastic region 70 of containment flaps 50, 52 can be of a longitudinal length that is less than the length of the absorbent article 10, 210. In this exemplary method of bonding the elastic members 68 to the containment flaps 50, 52, the portion of the elastic members 68 not coated with adhesive, will retract after the elastic members 68 and the absorbent article 10 are cut in manufacturing to form an individual absorbent article 10. As noted above, the relaxing of the elastic members 68 in the active flap elastic region 70 when the absorbent article 10, 210 is in a relaxed condition can cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28), as depicted in FIG. 5. Of course, the elastic members 68 can be bonded to the containment flaps 50, 52 in various other ways as known by those of skill in the art to provide an active flap elastic region 70, which is within the scope of this disclosure.

Where the elastic members 68 of the containment flaps are not present when the article 10, 210 is in a stretched position, the containment flaps comprise one or more inactive regions, such as zones 73. Some embodiments comprise such inactive zones 73 extending from the front waist edge 22 and the rear waist edge 24, with the active zone 70 disposed between these two inactive regions. Typically, the active zone 70 may have a length of between about 70%-80% of an overall article length. Although, as will be described in more detail below, embodiments according to aspects of the present disclosure may have active flap zones 70 which have a length of between about 45%-75%, or between 50% and 75%, or between 50% and 70%, or between 50% and 60% of an overall article length.

The containment flaps 50, 52 preferably have an elastic tension, as determined by the Elastic Tension Test Method described herein, of between 40 gf and 200 gf. The elastic tensions of the containment flaps 50, 52 can have an impact on fit and comfort as well as leakage. In accordance with embodiments of the present disclosure, the elastic tensions of the containment flaps 50, 52 also play a role in the presence and/or prevalence of creases and folds in articles 10, 210. For example, as discussed, the thin and flexible absorbent articles 10, 210 of the present disclosure are more prone to creasing and folding under the contraction forces of the containment flaps 50, 52. According to some embodiments of the present disclosure, elastic tensions of the containment flaps 50, 52 as low as 40 gf have been insufficient to prevent creases or folds from forming in absorbent articles 10, 210. Elastic tensions as high as 200 gf are useful for helping to ensure a tight seal on a wearer and to prevent leaks. However, certain wearers can experience uncomfortable tightness or skin red-marking with containment flaps 50, 52 having such elastic tensions. It has been found that a preferred range for the elastic tensions of the containment flaps 50, 52 is between about 50 gf and about 175 gf, or between about 60 gf and about 150 gf, or between about 75 gf and about 125 gf, or between about 75 gf and about 105 gf. Such ranges of elastic tensions of the containment flaps provide good balance between leakage performance and wearer comfort and still allow the articles 10, 210 of the present disclosure to resist the retractive forces of the flaps 50, 52 so that no creases or folds form in the absorbent articles 10, 210 when in a relaxed configuration-even when the absorbent articles 10, 210 are as thin and flexible as described herein.

### Leg Elastics:

Leg elastic members 60, 62 can be secured to the outer cover 26, such as by being bonded thereto by laminate adhesive, generally laterally inward of the longitudinal side edges, 18 and 20, of the absorbent article 10, 210. The leg elastic members 60, 62 can form elasticized leg cuffs that further help to contain body exudates. In an embodiment, the leg elastic members 60, 62 may be disposed between inner and outer layers (not shown) of the outer cover 26 or between other layers of the absorbent article 10, for example, between the base portion 64 of each containment flap 50, 52 and the bodyside liner 28 as depicted in FIG. 5, between the base portion 64 of each containment flap 50, 52 and the outer cover 26, or between the bodyside liner 28 and the outer cover 26. The leg elastic members 60, 62 can be one or more elastic components near each longitudinal side edge 18, 20. For example, the leg elastic members 60, 62 as illustrated herein each include two elastic strands. A wide variety of elastomeric materials may be used for the leg elastic members 60, 62.

Suitable elastomeric materials can include sheets, strands or ribbons of natural rubber, synthetic rubber, or thermoplastic elastomeric materials. The elastomeric materials can be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 can be formed with the containment flaps 50, 52, and then attached to the chassis 11 in some embodiments. Of course, the leg elastic members 60, 62 can be omitted from the absorbent article 10, 210 without departing from the scope of this disclosure.

The leg elastic members 60, 62 typically define a leg elastic active zone 77 of the leg cuffs. For example, as shown in FIGS. 2 and 4, the leg elastic members 60, 62 are shown as disposed along a certain portion of an overall length of the article 10, 210. The portion where the leg elastic members 60, 62 are present define the leg elastic active zone 77. The portion of the leg cuffs where the leg elastic members 60, 62 are absent are the leg cuff inactive zones 81. In some embodiments, the leg elastic active region may be between 50% and 90% of an overall article 10, 210 longitudinal length. In more specific embodiments, the leg elastic active region may be between 50% and 80%, or between 50% and 75%, or between 50% and 70%, or between 50% and 65% of an overall article 10, 210 longitudinal length.

### Waist Containment Member:

In an embodiment, the absorbent article 10, 210 can have one or more waist containment members 54. The waist containment member(s) 54 can be disposed in the rear waist region 14 as illustrated in FIGS. 1-5. In general, the waist containment member 54 can help contain and/or absorb body exudates, especially low viscosity fecal matter, and as such, can be preferred to be in the rear waist region 14. In some embodiments, the absorbent article 10, 210 can have a waist containment member 54 disposed in the front waist region 12. A waist containment member 54 in the front waist region 12 can help contain and/or absorb body exudates, such as urine, in the front waist region 12. Although not as prevalent as in the rear waist region 14, in some circumstances, fecal material may also spread to the front waist region 12, and thus, a waist containment member 54 disposed in the front waist region 12 can help contain and/or absorb body exudates as well. In other embodiments, the absorbent article 10, 210 can have a waist containment member 54 in both the rear waist region 14 and the front waist region 12.

The waist containment member 54 can be disposed on the body facing surface 45 of the absorbent assembly 44. In some embodiments, such as in embodiments illustrated in FIGS. 1-2 and 5, the waist containment member 54 can be disposed on the body facing surface 56 of the bodyside liner 28. However, in some embodiments, such as the absorbent article 210 in FIG. 4, the waist containment member 54 can be disposed on a body facing surface 58 of the rear waist panel 15.

The waist containment member 54 can include a first longitudinal side edge 72 and a second longitudinal side edge 74. The first longitudinal side edge 72 can be opposite from the second longitudinal side edge 74. The distance between the first longitudinal side edge 72 and the second longitudinal side edge 74 can define a width 51 of the waist containment member 54 in the lateral direction 32, as shown in FIG. 2.

As illustrated in FIGS. 2 and 5, the waist containment member 54 can be configured such that the first longitudinal side edge 72 can be disposed laterally outward of the proximal end 64a of the base portion 64 of the containment flap 50. Similarly, the waist containment member 54 can be configured such that the second longitudinal side edge 74 can be disposed laterally outward of the proximal end 64a of the base portion 64 of the containment flap 52. The waist containment member 54 can be configured such that the width 51 of the waist containment member 54 can be greater than a lateral distance between longitudinal extending fold lines 25a, 25b, as shown in FIGS. 2 and 4.

The waist containment member 54 can also include a proximal portion (not shown) and a distal portion 78. The proximal portion can be coupled to the body facing surface 19 of chassis 11 (e.g., the body facing surface 45 of the absorbent assembly 44 or the body facing surface 56 of the bodyside liner 28) whereas the distal portion 78 of the waist containment member 54 can be free to move with respect to the chassis 11 and the absorbent assembly 44 when the absorbent article 10, 210 is in the relaxed configuration, such as shown in FIG. 5. When the waist containment member 54 is in a relaxed configuration, the distal portion 78 extends away from the chassis 11 and absorbent assembly 44 in a vertical direction, which is perpendicular to the plane defined by the longitudinal axis 29 and the lateral axis 31. A fold 79a can separate the proximal portion from the distal portion 78 of the waist containment member 54. As used in this context, the fold 79a separates the proximal portion from the distal portion 78 in that the fold 79a defines a transition between the proximal portion and the distal portion 78.

In some embodiments, the proximal portion of the waist containment member 54 can be coupled to the body facing surface 56 of the bodyside liner 28. In other embodiments, the proximal portion of the waist containment member 54 can be coupled to the body facing surface 58 of the rear waist panel 15. The proximal portion can be coupled to the body facing surface 45 by an adhesive, by pressure bonding, by ultrasonic bonding, by thermal bonding, and combinations thereof.

Because the distal portion 78 of the waist containment member 54 can freely move with respect to the absorbent assembly 44 when the absorbent article 10, 210 is in the relaxed configuration, the distal portion 78 can help provide a containment pocket 82 when the absorbent article 10, 210 is in the relaxed configuration. The containment pocket 82 can help provide a barrier to contain and/or can help absorb body exudates. The containment pocket 82 can be especially beneficial for containing and/or absorbing low viscosity fecal matter, which can be prevalent in younger children. The first longitudinal side edge 72 can be disposed laterally outward of the proximal end 64a of the base portion 64 of the containment flap 50, and thus, the containment pocket 82 can extend laterally outward of the proximal end 64a of the containment flap 50. Similarly, the second longitudinal side edge 74 can be disposed laterally outward of the proximal end 64a of the base portion 64 of the containment flap 52 and the containment pocket 82 can extend laterally outward of the proximal end 64a of the containment flap 52. Such a configuration provides waist containment member 54 with a wide containment pocket 82 to contain and/or absorb body exudates.

To help prevent lateral flow of body exudates that are contained by the containment pocket 82 of the waist containment member 54, the distal portion 78 of the waist containment member 54 can be bonded to the proximal portion of the waist containment member 54 and/or the body facing surface 19 of the chassis 11 near the first and second longitudinal side edges 72, 74, respectively. For example, FIG. 5 depicts tack-down regions 84 where the distal portion 78 of the waist containment member 54 can be bonded to the proximal portion of the waist containment member 54 and/or the body facing surface 19 of the chassis 11.

In preferred embodiments, the waist containment member 54 can include at least one elastic member and even more elastic members in further embodiments. Generally, the elastic member can span substantially from the first longitudinal side edge 72 to the second longitudinal side edge 74 of the waist containment member 54. The elastic member can be disposed in the distal portion 78 of the waist containment member 54, and preferably, is located near a free edge 88 of the distal portion 78 of the waist containment member 54.

A wide variety of elastomeric materials may be used for the elastic member(s) in the waist containment member 54. Suitable elastomeric materials can include sheets, strands or ribbons of natural rubber, synthetic rubber, elastic foams, or thermoplastic elastomeric materials (e.g., films). The elastomeric materials can be stretched and secured to a substrate forming the waist containment member 54, secured to a gathered substrate, or secured to a substrate and then elasticized or shrunk, for example, with the application of heat, such that the elastic retractive forces are imparted to the substrate forming the waist containment member 54.

The waist containment member 54 can be disposed to be coupled to the chassis 11 by being placed either over the containment flaps 50, 52 or under the containment flaps 50, 52. More specifically, the waist containment member 54 can be disposed on the body facing surface 19 of the chassis 11 such that the proximal portion of the waist containment member 54 is disposed over the base portion 64 of the first and the second containment flaps 50, 52, respectively. Alternatively, the waist containment member 54 can be disposed on the body facing surface 19 of the chassis 11 such that the proximal portion of the waist containment member 54 is disposed under the base portion 64 of the first and the second containment flaps 50, 52, respectively. Both configurations can provide advantages to the functioning of the waist containment member 54 to contain and/or absorb body exudates.

Where the proximal portion of the waist containment member 54 is disposed over the base portion 64 of the containment flaps 50, 52, the containment flaps 50, 52 can have an active flap elastic region 70 that longitudinally overlaps with the distal portion 78 of the waist containment member 54 when the absorbent article 10 is in the stretched, laid flat configuration, such as illustrated in FIG. 2. Additionally or alternatively, the tack-down region 71 may not extend from the rear waist edge 24 to the free edge 88 of the distal portion 78 of the waist containment member 54, such as illustrated in FIG. 2.

Where the proximal portion of the waist containment member 54 is disposed under the base portion 64 of the containment flaps 50, 52, the tack-down region 71 of the projection portion 66 of each of the containment flaps 50, 52 may longitudinally overlap with the distal portion 78 of the waist containment member 54. In some of these embodiments, the tack-down region 71 of projection portion 66 of each of the containment flaps 50, 52 can extend to the free edge 88 of the waist containment member 54 to further assist in containing exudates to the containment pocket 82 created by the waist containment member 54.

The waist containment member 54 can be comprised of a variety of materials. In a preferred embodiment, the waist containment member 54 can be comprised of a spunbond-meltblown-spunbond (“SMS”) material. However it is contemplated that the waist containment member 54 can be comprised of other materials including, but not limited to, a spunbond-film-spunbond (“SFS”), a bonded carded web (“BCW”), or any non-woven material. In some embodiments, the waist containment member 54 can be comprised of a laminate of more than one of these exemplary materials, or other materials. In some embodiments, the waist containment member 54 can be comprised of a liquid impermeable material. In some embodiments, the waist containment member 54 can be comprised of a material coated with a hydrophobic coating. The basis weight of the material forming the waist containment member 54 can vary, however, in a preferred embodiment, the basis weight can be between 8 gsm to 120 gsm, not including the elastic members 86 in the waist containment member 54. More preferably, the basis weight of the material comprising the waist containment member 54 can be between 10 gsm to 40 gsm, and even more preferably, between 15 gsm to 25 gsm.

### Fastening System:

In an embodiment, the absorbent article 10 can include a fastening system. The fastening system can include one or more back fasteners 91 and one or more front fasteners 92. The embodiments shown in FIGS. 1 and 2 depict embodiments with one front fastener 92. Portions of the fastening system may be included in the front waist region 12, rear waist region 14, or both.

The fastening system can be configured to secure the absorbent article 10 the waist of the wearer in a fastened condition as shown in FIG. 1 and help maintain the absorbent article 10 in place during use. In an embodiment, the back fasteners 91 can include one or more materials bonded together to form a composite ear as is known in the art. For example, the composite fastener may be composed of a stretch component 94, a nonwoven carrier or hook base 96, and a fastening component 98, as labeled in FIG. 2. As shown in FIG. 5, in some embodiments the waist containment member 54 can extend to back fasteners 91. In some embodiments, the waist containment member 54 can be coupled to the stretch component 94 of the back fasteners 91, either directly or indirectly. In some embodiments, the waist containment member 54 can extend to the longitudinal side edges 18, 20 of the absorbent article 10, 210.

### Absorbent Body:

The absorbent system 34 can be suitably constructed to be generally compressible, conformable, pliable, non-irritating to the wearer's skin and capable of absorbing and retaining liquid body exudates. The absorbent system 34 can be manufactured in a wide variety of sizes and shapes (for example, rectangular, trapezoidal, T-shape, I-shape, hourglass shape, etc.) and from a wide variety of materials. The size and the absorbent capacity of the absorbent system 34 should be compatible with the size of the intended wearer (infants to adults) and the liquid loading imparted by the intended use of the absorbent article 10, 210. The absorbent system 34 can have a length and width that can be less than or equal to the length and width of the absorbent article 10, 210.

If a spacer layer 48 is present, the absorbent system 34 can be disposed on the spacer layer 48 and superposed over the outer cover 26. The spacer layer 48 can be bonded to the outer cover 26, for example, by adhesive. In some embodiments, a spacer layer 48 may not be present and the absorbent system 34 can directly contact the outer cover 26 and can be directly bonded to the outer cover 26. However, it is to be understood that the absorbent system 34 may be in contact with, and not bonded with, the outer cover 26 and remain within the scope of this disclosure. In an embodiment, the outer cover 26 can be composed of a single layer and the absorbent system 34 can be in contact with the singer layer of the outer cover 26. In some embodiments, at least a portion of a layer, such as but not limited to, a fluid transfer layer 46 and/or a spacer layer 48, can be positioned between the absorbent system 34 and the outer cover 26, such as illustrated in FIG. 5. The absorbent system 34 can be bonded to the fluid transfer layer 46 and/or the spacer layer 48. In an embodiment, the absorbent system 34 can be composed of absorbent material, such as fibrous absorbent material and/or, superabsorbent material, binder materials, surfactants, selected hydrophobic and hydrophilic materials, pigments, lotions, odor control agents or the like, as well as combinations thereof. In an embodiment, the absorbent system 34 can be a matrix of cellulosic fluff and superabsorbent material. In another embodiment, the absorbent material of the absorbent system 34 can comprise only superabsorbent material. When composed at least partially of superabsorbent materials, such superabsorbent materials can be selected from natural, synthetic, and modified natural polymers and materials. The superabsorbent materials can be inorganic materials, such as silica gels, or organic compounds, such as cross-linked polymers.

In an embodiment, the absorbent system 34 may be constructed of a single layer of materials, or in the alternative, may be constructed of two or more layers of materials. Where the absorbent system 34 is comprised of multiple layers of absorbent material, the combination of the absorbent layers forms the absorbent system 34. Further, absorbent systems 34 of contemplated embodiments may have particular properties that aide the articles 10, 210 of the present disclosure to have desirable fit and comfort properties. In such embodiments, the absorbent system 34 may comprise a high content of superabsorbent material. For example, preferred absorbent systems 34 may have the superabsorbent material comprise greater than about 80% by weight of the total weight of absorbent material of the absorbent system 34. In further embodiments, the superabsorbent material may comprise greater than about 85% by weight, or greater than about 90% by weight, or greater than about 95% by weight, or up to 100% by weight of the total weight of absorbent material of the absorbent system 34. Such absorbent systems 34 may further comprise an adhesive to immobilize the superabsorbent material. The adhesive may be present in an amount between about 2% and about 10%, or more preferably between about 3% and about 8%, or between about 3% and about 7%, or between about 3% and about 6%, or between about 3% and 5%. The superabsorbent material and adhesive may be disposed between corewrap materials having basis weights of between about 10 gsm and about 75 gsm, or between about 10 gsm and about 65 gsm, or between about 10 gsm and about 55 gsm, or between about 10 gsm and about 45 gsm. In some particular embodiments, the absorbent system 34 may be formed of one or more absorbent bodies 101 as described in PCT Application No. PCT/US2020/047741, to Schoon et al.

These absorbent systems 34 may be formed of superabsorbent material having a basis weight of between about 200 gsm and about 500 gsm. In other embodiments, the absorbent systems 34 may be formed of superabsorbent material having a basis weight of between about 250 gsm and about 450 gsm, or between about 250 gsm and about 400 gsm. In at least some embodiments, the absorbent system 34 of the present disclosure may have a relatively uniform distribution of superabsorbent material. For example, the absorbent systems 34 may be notionally divided into a front region 34a, a middle region 34b, and a rear region 34c, as shown in FIGS. 2 and 4. Each of the front region 34a, the middle region 34b, and the rear region 34c may comprise a third of an overall longitudinal length of the absorbent system 34. According to aspects of the present disclosure, an average basis weight any one of the front region 34a, the middle region 34b, and the rear region 34c may not vary by more than about 20% of an average basis weight of any other one of the front region 34a, the middle region 34b, and the rear region 34c. As one illustrative example, if the average basis weight of the front region 34a is 250 gsm, the middle region 34b and the rear region 34c may not have average basis weights of less than approximately 208 gsm or more than about 300 gsm. In further embodiments, an average basis weight any one of the front region 34a, the middle region 34b, and the rear region 34c may not vary by more than about 15%, or vary by not more than about 10%, or vary by not more than about 5% of an average basis weight of any other one of the front region 34a, the middle region 34b, and the rear region 34c.

Such absorbent systems 34 as described above may be particularly thin and flexible, and as such provide good fit and comfort qualities for a wearer. For instance, embodiments according to the present disclosure with such above-described absorbent systems 34 can provide for very thin absorbent systems, such as less than about 5 mm, or even less than about 4.5 mm, or even less than about 4.0 mm, or even less than about 3.5 mm, or even less than about 3.0 mm, or even less than about 2.5 mm, or even less than about 2.0 mm. Such thin absorbent systems 34 help to improve comfort or a wearer. Additionally, where such absorbent systems 34 have relatively uniform basis weights of superabsorbent material as described above, the articles 10, 210 of the present disclosure can provide for improved comfort for a wearer by not forming bulges in the article after liquid uptake or by applying different pressure to a wearer's body after liquid uptake due to large differences in superabsorbent material distribution.

As well, such absorbent systems 34 may provide for greatly improved flexibility of the absorbent systems 34 as compared to other absorbent systems having greater amounts of non-superabsorbent absorbent material (such as pulp fluff or the like) and/or having differing properties than described above. For example, when used within articles 10, 210 of the present disclosure, portions of the articles 10, 210 having absorbent systems 34 have been able to achieve probe flexibility measurements of less than 60 gf, less than 50 gf, less than 40 gf, less than 35 gf, less than 30 gf, less than 25 gf, less than 20 gf, and even less than 15 gf.

Although products with such probe flexibility measurements may provide for an improved fit and comfort for a wearer, such high flexibilities can impact other aspects of articles 10, 210 in undesirable manners. For example, where articles 10, 210 have probe flexibility measurements of less than about 60 gf, elastic tensions within the article may cause the articles 10, 210 to form undesirable creases or folds. For example, the articles 10, 210 may not be able to resist the retractive forces of the active regions of the containment flaps and/or the leg elastics to a sufficient degree to prevent the elastic members from buckling the articles 10, 210 causing creases and/or folds.

Such creases and/or folds may be particularly undesirable in articles comprising outer cover graphics. For example, as shown in FIG. 1, article 10 may comprise a graphical feature 121 disposed on the outer cover 26. In some embodiments, the graphical feature 121 is a continuous graphic feature comprising at least one cohesive theme or at least one individual graphical element that extends from the front or rear waist regions 12, 14 into the crotch region 16. The presence of one or more creases and/or folds can distort the graphical feature 121 in an undesirable manner. As one illustrative example, the graphical feature 121 can comprise a singular graphical element comprising an anthropomorphous component extending from the front or rear waist regions 12, 14 into the crotch region 16. The presence of one or more creases or folds—particularly to a head or upper torso region of the anthropomorphous component—can cause undesirable distortions to the anthropomorphous component. In other embodiments, the formation of creases and/or folds can occur, occur more frequently, or occur to a greater degree (e.g. be larger or more numerous) where the articles 10, 210 have probe flexibility measurements of less than about 50 gf, or less than about 40 gf, or less than about 35 gf, or less than about 30 gf, or less than about 25 gf, or less than about 20 gf, or less than about 15 gf. Such problems can occur particularly when the elastic members 68 of the active regions of the containment flaps 50, 52 and/or the elastic members 60, 62 of the leg cuffs have individual tensions of between 40 gf and 200 gf.

Articles 10, 210 of the present disclosure have been able to maintain desirable flexibilities while mitigating against such crease and/or folding issues in a surprising way. It has been found that aligning the active regions 70 of the containment flaps 50, 52 and/or the active regions of the leg cuffs, defined by leg elastic members 60, 62 with portions of the articles 10, 210 can prevent such creasing and/or folding, as will be described in more detail below.

In particular embodiments according to aspects of the present disclosure, articles 10, 210 have absorbent systems 34 comprising greater than 80%, greater than 90%, or up to 100% superabsorbent material by total weight of absorbent material of the absorbent systems 34. The absorbent systems 34 may also have a relatively uniform distribution of superabsorbent material. According to some embodiments, an average basis weight any one of the front region 34a, the middle region 34b, and the rear region 34c may not vary by more than about 20%, or more than about 10%, or more than about 5% of an average basis weight of any other one of the front region 34a, the middle region 34b, and the rear region 34c. The absorbent systems 34 may additionally have thicknesses of less than about 4 mm, or less than about 3.5 mm, or less than about 3.0 mm, or less than about 2.5 mm, or less than about 2.0 mm. The absorbent systems 34 may generally have a thickness of greater than about 1.0 mm. These thicknesses were determined according to the Thickness Determination Test Method described herein.

These articles 10, 210 may further have containment flap active zones 70 and/or leg cuff active zones 77 that extend for between 45% and 80%, or between 45% and 70%, or between 45% and 62.5%, or between 45% and 55% of an overall length of the articles 10, 210. These active zones 70, 77 may be disposed between inactive zones 73 or 81 disposed proximate the front waist edge 22 and the rear waist edge 24. The inactive zones 73, 81 disposed proximate the front waist edge 22 may extend from the front waist edge 22 toward the rear waist edge and may have a length of between 10% and 30%, or between 10% and 25%, or between 10% and 20% of the overall length of the articles 10, 210. These active zones 70, 77 may define active regions of the articles 10, 210 covering the entire lateral width of the articles 10, 210 within the area of the active zones 70, 77. The elastic members 68 and/or 60, 62 may be applied to the articles 10, 210 having tensions of between 40 gf and 200 gf, or between 50 gf and 150 gf, or between 75 gf and 105 gf. Tensions in this range help to ensure the containment flaps 50, 52 and the leg cuffs have sufficient tension to gasket a wearer's body and help prevent leaks as well as ensure a comfortable fit.

The articles 10, 210 may further include a fluid acquisition layer 43 disposed above the absorbent system 34. For example, the fluid acquisition layer 43 may be disposed between the absorbent system 34 and the bodyside liner 34 as in FIG. 5. Although, in other embodiments, the fluid acquisition layer 43 may be a topical material applied to the bodyside liner 28 on the body facing surface 19 of the chassis. The fluid acquisition layer 43 may comprise a single material or a multi-layer material comprising a fibrous nonwoven web(s). The fluid acquisition layer 43 may be designed to quickly intake liquid and distribute the liquid throughout the fluid acquisition layer 43. Example nonwoven web(s) include spunbond webs, meltblown webs, and staple fiber webs such as through air bonded carded webs (TABCWs) and hydroentangled webs. Such fluid acquisition layer 43 are typically formed of polymer fibers. In some embodiments, where the fluid acquisition layer 43 is not inherently hydrophilic, the fluid acquisition layer 43—or the fibers forming the fluid acquisition layer 43—are treated to be hydrophilic. Useful basis weights for the fluid acquisition layer 43 are generally between about 15 gsm and about 150 gsm, or more specifically between about 20 gsm and about 125 gsm, or between about 30 gsm and 100 gsm.

It has surprisingly been found that presence or absence of the fluid acquisition layer 43 can significantly impact the flexibility of the articles 10, 210—even to a degree where the articles 10, 210 are stiff enough to resist the forces of the elastic members 68 of the containment flaps 50, 52 and/or the leg elastic members 60, 62 and prevent creasing and/or folding of the articles 10, 210. According to some embodiments of the present disclosure, the articles 10, 210 may comprise first locations within the inactive regions (e.g. portions of the articles 10, 210 encompassing the inactive zones 73, 81 of the containment flaps 50, 52 and the leg cuffs) where the absorbent system is present which has a probe flexibility of less than or equal to 40 gf. Such first locations, sample preparation of such first locations, and the method to determine a probe flexibility are detailed below with respect to the Probe Flexibility Test Method. In further embodiments, such first locations may have a probe flexibility of less than or equal to 35 gf, or less than or equal to 30 gf, or less than or equal to 25 gf, or less than or equal to 20 gf, or less than or equal to 15 gf. In general, such first locations may have a probe flexibility of greater than 5 gf. Typically, the fluid acquisition layer 43 is not present at such first locations—although, this feature is not required. These high flexibilities (e.g. low values for the probe flexibility measurement) are desirable from a fit and comfort perspective.

These embodiments of the articles 10, 210 may comprise second locations disposed within the active regions (e.g. portions of the articles 10, 210 encompassing the active zones 70, 77 of the containment flaps 50, 52 and the leg cuffs) where the absorbent system is present and where the fluid acquisition layer 43 is present. Such second locations, sample preparation of such second locations, and the method to determine a probe flexibility are detailed below with respect to the Probe Flexibility Test Method. The second locations may desirably have a probe flexibility of greater than or equal to 40 gf. In further embodiments, such second locations may have a probe flexibility of greater than or equal to 45 gf, or greater than or equal to 50 gf, or greater than or equal to 55 gf, or greater than or equal to 60 gf, or greater than or equal to 65 gf. In general, such second locations may have a probe flexibility of less than 100 gf. It has been found that such flexibility values for second locations are sufficient to prevent the undesired creasing and/or folding of the articles 10, 210 having such thin and flexible absorbent systems 34.

One of the surprising findings is that the fluid acquisition layer 43 is able to increase the stiffness (e.g. reduce flexibility) of the articles 10, 210 without adding much thickness to the articles 10, 210. For instance, if the articles 10, 210 may be thought of as a beam, thickness contributes in a cubic fashion to buckling of the beam (e.g. causing a crease or fold). However, the articles 10, 210 of the present disclosure may have article first thicknesses at the first locations and second thicknesses at the second locations which do not greatly differ. In measurements of the articles 10, 210 of the present disclosure, the thicknesses measured at the second locations may not be greater than the first thickness by more than 20% of the first thickness. In further embodiments, articles 10, 210 were measured to have the second thickness not be greater than the first thickness by more than 10% of the first thickness. In still further embodiments, articles 10, 210 were measured to have the second thickness not be greater than the first thickness by more than 5% of the first thickness. In each of these examples, the articles 10, 210 exhibited sufficient stiffness in the second locations to reduce and/or eliminate the creasing or folding problems. Both the first and second thicknesses may be less than about 6 mm, or less than about 5.5 mm, or less than about 5.0 mm, or less than about 4.5 mm. All of these described thicknesses were determined according to the Thickness Determination Test Method described herein.

In contrast to the low variation in thickness between the first location and the second location, the presence or absence of the fluid acquisition layer 43 can affect the probe flexibility measurement by over 400% As can be seen in Table 1 below, an example product had a probe flexibility at a first location of approximately 15 gf, while having a probe flexibility at a second location of over 60 gf. In some embodiments, the fluid acquisition layer 43 may be bonded to one or more other layers within the second location. For example, the fluid acquisition layer 43 may be bonded to the absorbent system 34 and/or the bodyside liner 28 by adhesive, ultrasonic bonding, and/or pressure and/or heat bonding. Where the fluid acquisition layer 43 is bonded to the absorbent system 34 and/or the bodyside liner 28 by adhesive, the adhesive may be present at an amount of greater than or equal to 0.3 gsm and less than or equal to 5 gsm. Where the fluid acquisition layer 43 is bonded to the absorbent system 34 and/or the bodyside liner 28, it is believed that such bonding may further enhance the stiffness of the article at such locations.

Accordingly, it is believed that particular combinations of flexibility (as measured according to the Probe Flexibility Test Method described herein) and thickness are particularly useful to ensure creases and folds do not form in the articles 10, 210 when in a relaxed configuration. For example, it is believed that the articles 10, 210 in the active regions 70 should have a combination of flexibility and thickness such that the flexibility multiplied by the cube of the thickness is greater than or equal to 2534 (gf*mm³).

In order to take advantage of this finding, particular embodiments of articles 10, 210 of the present disclosure may place the fluid acquisition layer 43 in particular locations relative to other portions of the articles 10, 210. According to some embodiments, the fluid acquisition layer 43 has a front edge disposed proximate the article front edge 22 and a rear edge disposed proximate the article rear edge 24. The fluid acquisition layer 43 front edge may be positioned relative to the active zone 70 of the containment flaps 50, 52 such that the fluid acquisition layer 43 front edge is disposed no closer to the article front edge 22 than the active zone 70 of the containment flaps 50, 52. In at least some of these embodiments, the absorbent system 34 first end edge 40 is disposed closer to the article front waist edge 22 than the fluid acquisition layer 43 front edge and is disposed within the inactive region of the article encompassing the inactive zone 73 disposed between the containment flaps active zone 70 and the article front waist edge 22. In such embodiments, since the contraction force of the active zone 70 of the containment flaps 50, 52 begins where the articles 10, 210 have sufficient stiffness (e.g. reduced flexibility as compared to locations of the article within the inactive regions) to resist the forces of the elastic members 68 and prevent a crease and/or fold from forming in the articles 10, 210.

In further refined embodiments, the leg cuff active zone 77 may begin at the same longitudinal position within the article 10, 210 as the containment flap active zone 77, with respect to the article front waist edge 22. In alternative of these embodiments, the leg cuff active zone 77 may be disposed further away from the article front waist edge 22 than the containment flap active zone 77.

According to further embodiments of the present disclosure, the articles 10, 210 may comprise a graphical feature 121 disposed at least in the front region 12. The graphical feature may extend continuously in some embodiments between the front region 12 and the crotch region 16. The graphical feature 121 may be formed of a singular theme or motif, and therefore it may be desired that the articles 10, 210 do not form any creases and or folds extending through the graphical feature 121 which would disrupt the theme or motif or distort the graphical feature 121 in an undesirable fashion. In some particular embodiments, the graphical feature 121 is an anthropomorphous element. The graphical feature 121 may further extend within the inactive region of the articles 10, 210 proximate the article front waist edge 22 defined by the inactive zones 73 of the containment flaps 50, 52 disposed between the active zones 70 and the article front waist edge 22. The graphical feature 121 may further extend through the transition between the inactive region and the active region of the articles 10, 210 and into the active region. Further, the graphical feature 121 may extend through the transition between the inactive region and the active region such that the graphical feature is unbroken by creases or folds of the articles 10, 210 when in a relaxed state.

The above description of the articles 10, 210 generally describes embodiments related to features proximate the article front waist edge 22 which can solve a problem of crease of fold formation in thin, flexible articles. However, it should be understood that a similar problem may be realized in portions of such articles 10, 210 proximate the rear waist edge 24. Accordingly, similar solutions and features of such articles 10, 210 apply equally to portions proximate the rear waist edge 24. For example, the first location may be chosen within the inactive region of the articles 10, 210 proximate the rear waist edge 24 instead of the front waist edge 22. Likewise, positioning of a rear edge of the fluid acquisition layer 43 in relation to the active zones 70 of the containment flaps 50, 52 and the rear waist edge 24 may be chosen in a similar fashion as described with respect to the front edge of the fluid acquisition layer 43 in relation to the active zones 70 of the containment flaps 50, 52 and the front waist edge 22.

Below are measurements taken from example articles 10, 210 as well as comparative absorbent articles comprising an absorbent system comprising mixtures of superabsorbent material and pulp fluff with the amount of superabsorbent material in a range below that of the example articles 10, 210. In these examples, Examples 1a-d are consistent with the articles 10 of the present disclosure. Examples 1a-d had a single-layer absorbent system 34 comprising an approximately uniform basis weight of 100% superabsorbent material by total weight of absorbent material. The absorbent system had the superabsorbent material present in an amount of about 340 gsm with about 7% adhesive weight, by total weight of the superabsorbent material. The fluid acquisition layer was disposed between the bodyside liner and the absorbent system and was comprised of an 85 gsm TABCW material and was positioned different distances away from the absorbent system front edge. Examples 2a,b is consistent with the articles 210 of the present disclosure and having the same absorbent system 34 as Examples 1a-d. Examples 2a,b had a fluid acquisition layer disposed on top of the bodyside liner and was comprised of a 75 gsm hydroentangled material.

The Comparative Example 1 is consistent with a Pull-Ups New Leaf® commercial product, size 4, purchased in 2022. The absorbent system comprised superabsorbent material at a weight of 56% by total weight of absorbent material, with the remaining 44% weight being pulp fluff. The fluid acquisition layer comprised an 85 gsm TABCW material.

TABLE 1

| | | Probe | Crease or Fold |
|---|---|---|---|
| | Measurement Location and Measurement | Flexibility | Observed in |
| CODE | Sample Contents | Measurement | Assembled Product |
| Comparative | Measurement was chosen at a first location | 60.7 gf | No/acceptable |
| Example 1 | where the absorbent system was present | | appearance |
| | but no fluid acquisition layer was present | | |
| | anywhere in the sample | | |
| Example 1a | Sample was chosen at a second location | 63.7 gf | No/acceptable |
| | and included both absorbent system and | | appearance |
| | fluid acquisition layer throughout the entire | | |
| | sample | | |
| Example 1b | Sample was chosen at a first location | 39.6 gf | No/acceptable |
| | aligned with a front edge of the absorbent | | appearance |
| | system, where the front edge of the fluid | | |
| | acquisition layer was disposed 10 mm | | |
| | away from the absorbent system front edge | | |
| Example 1c | Sample was chosen at a first location | 17.4 gf | Yes/unacceptable |
| | aligned with a front edge of the absorbent | | appearance |
| | system, where the front edge of the fluid | | |
| | acquisition layer was disposed 34 mm | | |
| | away from the absorbent system front edge | | |
| Example 1d | Sample was chosen at a first location | 15.0 gf | Yes/unacceptable |
| | aligned with a front edge of the absorbent | | appearance |
| | system, where the front edge of the fluid | | |
| | acquisition layer was disposed 58 mm | | |
| | away from the absorbent system front edge | | |
| | such that no fluid acquisition layer was | | |
| | present in the sample | | |
| Example 2a | Sample was chosen at a second location | 63.0 gf | ~Acceptable |
| | and included both absorbent system and | | |
| | fluid acquisition layer throughout the entire | | |
| | sample | | |

TABLE 2 below details the thicknesses of the Examples 1 and 2 and the Comparative Examples 1a-d at the different first and second locations to highlight the minimal differences in thicknesses between the first and second locations where the probe flexibility was measured.

TABLE 2

| | | | Other | |
|---|---|---|---|---|
| | Measurement Location and | Absorbent | Component | Total |
| Code | Measurement Sample Contents | Thickness | Thickness | Thickness |
| Comparative | Measurement was chosen at a first | 4.12 mm | 2.17 mm | 6.30 mm |
| Example 1 | location where no fluid acquisition layer | | | |
| | was present anywhere in the sample | | | |
| Comparative | Sample was chosen at a second | 4.60 mm | 2.39 mm | 6.99 mm |
| Example 1 | location and included both absorbent | | | |
| | system and fluid acquisition layer | | | |
| | throughout the entire sample | | | |
| Example 1d | Measurement was chosen at a first | 1.47 mm | 2.49 mm | 3.96 mm |
| | location where no fluid acquisition layer | | | |
| | was present anywhere in the sample | | | |
| Example 1a | Sample was chosen at a second | 1.62 mm | 2.48 mm | 4.10 mm |
| | location and included both absorbent | | | |
| | system and fluid acquisition layer | | | |
| | throughout the entire sample | | | |
| Example 2a | Measurement was chosen at a first | 1.74 mm | 1.85 mm | 3.59 mm |
| | location where no fluid acquisition layer | | | |
| | was present anywhere in the sample | | | |
| Example 2b | Sample was chosen at a second | 2.20 mm | 1.91 mm | 4.11 mm |
| | location and included both absorbent | | | |
| | system and fluid acquisition layer | | | |
| | throughout the entire sample | | | |

TABLE 3

| | | Bending | Bending | Bending |
|---|---|---|---|---|
| | | Stiffness | Stiffness | Stiffness |
| | Measurement Location and | B+ (gf | B− (gf | Bmean (gf |
| Code | Measurement Sample Contents | cm²/cm) | cm²/cm) | cm²/cm) |
| Comparative | Measurement was chosen at a first | 20.9 | 17.8 | 19.4 |
| Example 1 | location where no fluid acquisition layer | | | |
| | was present anywhere in the sample | | | |
| Example 1b | Sample was chosen at a first location | 9.62 | 3.02 | 6.32 |
| | aligned with a front edge of the | | | |
| | absorbent system, where the front edge | | | |
| | of the fluid acquisition layer was | | | |
| | disposed 10 mm away from the | | | |
| | absorbent system front edge | | | |
| Example 1d | Measurement was chosen at a first | 8.73 | 2.14 | 5.43 |
| | location where no fluid acquisition layer | | | |
| | was present anywhere in the sample | | | |

TABLE 4

| | | Hysteresis | Hysteresis | Hysteresis |
|---|---|---|---|---|
| | Measurement Location and | 2HB+ (gf | 2HB− (gf | 2HBmean (gf |
| Code | Measurement Sample Contents | cm/cm) | cm/cm) | cm/cm) |
| Comparative | Measurement was chosen at a first | 3.85 | 4.61 | 4.23 |
| Example 1 | location where no fluid acquisition layer | | | |
| | was present anywhere in the sample | | | |
| Example 1b | Sample was chosen at a first location | 2.71 | 1.83 | 2.27 |
| | aligned with a front edge of the | | | |
| | absorbent system, where the front edge | | | |
| | of the fluid acquisition layer was | | | |
| | disposed 10 mm away from the | | | |
| | absorbent system front edge | | | |
| Example 1d | Measurement was chosen at a first | 2.66 | 2.23 | 2.44 |
| | location where no fluid acquisition layer | | | |
| | was present anywhere in the sample | | | |

TABLES 3 and 4 detail an alternative test of flexibility of the articles 10, 210 of the present disclosure-bending stiffness. The bending stiffness may be measured according to the bending stiffness test detailed within U.S. Patent Application Publication No. US2010/0158964 to Cunningham et al., the entirety of which is hereby incorporated by reference.

As can be seen, Example 1 performs superior to the Comparative Example 1. Additionally, it was found that the articles 10, 210 in the second locations should have a mean bending stiffness of greater than or equal to 5.43 to help alleviate issues related to creasing and folding. In some embodiments, the articles desirably have mean bending stiffnesses of greater than or equal to 5.5, greater than or equal to 5.75, greater than or equal to 5.9, greater than or equal to 6.1, or greater than or equal to 6.3. In such embodiments, the articles 10, 210 may desirably have mean bending stiffnesses less than or equal to 15, or less than or equal to 12.5, or less than or equal to 10, or less than or equal to 7.5, to ensure good flexibility for fit and comfort.

### Probe Flexibility Test Method:

This test method is a single-cycle compression bench test to measure the force required to deflect a segment of a material sample into a circular orifice to a fixed distance. The procedure measures load values when the material is pressed into the orifice by a probe. The peak load value is reported. This method is intended to be consistent with ASTM D4032 Standard Test Method for Stiffness of Fabric by the Circular Bend Procedure.

### Overview and Specimen Preparation:

A material sample is prepared by cutting specimens from sample products or materials. A die cut may be used for this purpose. Specimen dimension is a square 37.5 mm by 37.5 mm. Used to test articles 10, 210 of the present disclosure, the specimens are taken directly from an intact article. In these instances, the straight edges of the square should be aligned with the longitudinal and lateral axes of the articles. Where specimens possess multiple layers not bonded or joined together, special care should be taken to maintain the original relative orientation of layers, and to keep edges of all component layers flush.

Where the material sample is chosen from a ‘first location’ as described herein, the sample should have the absorbent core present throughout the entire sample. Some portions of these samples may further include a fluid acquisition layer. Where the material sample is chosen from a ‘second location’ as described herein, the sample should have both the absorbent core and the fluid acquisition layer present throughout the entire sample. If possible, the second location should be chosen such that the entire sample is cut from within the active zones 70.

Elastics that may be present in the article should be removed prior to obtaining specimens, taking care to avoid cutting, compressing, or distorting the absorbent core during removal. Alternatively, the specimens may be carefully chosen such that no. Specimens should be tested with the body-side of the absorbent (or product) facing the test probe.

### Apparatus and Materials:

Tensile tester: MTS tensile tester model Synergie 200 Test Bed; available from MTS® Systems Corporation, Research Triangle Park, North Carolina USA, or an equivalent system, preferably equipped with a computerized data-acquisition system capable of calculating a peak load in grams.

Load cells: A suitable cell selected so the majority of the peak load values fall between the manufacturer's recommended ranges of load cell's full scale value; for example, a 2,000 g load cell may be appropriate, such as may be obtained from MTS® Systems Corporation, Research Triangle Park, North Carolina USA. The load cell must be able to measure compression.

Operating software and data acquisition system: MTS TestWorks® for Windows software version 4; available from MTS® Systems Corporation, Research Triangle Park, North Carolina USA, or an equivalent system for the tensile tester used.

The apparatus employed is a modified Circular Bend Stiffness Tester, having the following parts: a smooth-polished metal plate platform which is 110 mm (length) by 102 mm (width) by 6.35 mm (depth) having a 18.75 mm diameter orifice. The lap edge of the orifice should be at a 45 degree angle to a depth of 4.75 mm.

A plunger having the following dimensions is used: overall length of 100 mm, a diameter of 6.25 mm, a ball nose having a radius of 2.97 mm and a needle-point extending about 1 mm from the ball nose with a 0.33 mm base diameter and a point having a radius of less than 0.5 mm. The plunger is mounted concentrically with the orifice having equal clearance on all sides. The needle-point is used merely to prevent lateral movement of a sample during testing. The bottom of the plunger should be set well above the top of the orifice plate. From this position, the downward stroke of the ball nose is to the exact bottom of the plate orifice.

### Sample Conditioning

Reasonable ambient conditions should be used for sample testing, such as 73+/−2 degrees Fahrenheit and a relative humidity of 50+/−2%. Samples should be allowed to equilibrate to laboratory conditions for at least two hours prior to testing.

### Apparatus Preparation and Procedure

### Tensile Tester Test Conditions:

Data acquisition rate 100 Hz

$$Crosshead speed 500 + / - 10 mm / min$$

Full scale load: 2,000 g
Gage length: 25.4+/−1 mm
Number of cycles: 1

### Procedure:

A. Center a specimen over the orifice.
B. Start the crosshead.
C. When the test is finished and the crosshead has returned, remove the specimen from the orifice.
D. Repeat the above steps for each additional specimen.
E. Continue testing all samples in this manner.
F. Report data for each sample in the following way:

### Average Peak Load

A specimen with a peak load that exceeds the limits of the load cell (^{˜}2,000 g) should have a peak load listed as >2,000 g. The average calculation for that sample should use 2,000 g as the peak load for that specimen, with a notation made that the average is conservative (low) due to rounding down at least one peak load level to 2,000 g.

### Elastic Tension Test Method:

To determine elastic tension with an article 10, 210, sample articles are hung from a conventional lightbox or other suitable device in an open, unfolded, vertical orientation with one end (e.g., the back end) of the article up and the body facing surface of the article facing laterally outward away from the lightbox. Open up the article if it is a closed product such as a pant article, cutting the pant side seam if it does not have mechanical fasteners.

To determine an elastic tension of containment flaps of an article, the lightbox should include a pair of fixed upper clamps spaced approximately 5.5 inches apart at their centerlines (e.g., spaced in accordance with the approximate spacing between the containment flaps on the opposite sides of the article). The waistband at the back end of the article is generally fully stretched (e.g., to eliminate gathers in or otherwise straighten the waistband) and the clamps are clamped to the waistband without clamping any of the absorbent core therein. The operator then gently runs his or her fingers down the sides of the containment flaps to straighten and extend the article.

The waistband at the front (i.e., lower) end of the article is then generally fully stretched (i.e., to eliminate gathers in or otherwise straighten the waistband) and a clamp weight is secured to the waistband at the front end of the article so that the article hangs freely from the upper clamps in a longitudinally elongated configuration. The clamp weight is approximately 1,000 grams total mass and includes a pair of clamps spaced approximately 5.5 inches apart at their centerlines (e.g., spaced approximately the same as the clamps attached to the back end of the article) for attaching the clamp weight to the front end of the article.

With the article in this longitudinally elongated configuration, a pair of markings is made on each containment flap generally where the elastics are attached to the flap. More particularly, the markings on each containment flap are spaced longitudinally from each other a distance of about 100 mm and are equidistant from the longitudinal center of the flap. The distance of 100 mm should encompass an area where the elastic members of the containment flaps are present. The center of the 100 mm distance may be adjusted to ensure each 100 mm distance comprises elastic members present for the entire distance. The clamp weight is then removed from the article and the article is removed from the lightbox. Each containment flap is cut from the article by first cutting the containment flap inward from the free (i.e., distal) edge of the flap to the secured edge of the flap at longitudinally spaced locations which are approximately 0.5 inches (12.85 mm) beyond each of the markings (e.g., toward the nearest end of the article) made on the flap, and then cutting lengthwise generally adjacent the bead of adhesive which secures the secured edge of the flap to the liner.

Each test specimen (e.g., the marked and cut portion of each containment flap) is then secured in a testing device by a generally fixed upper clip and a generally moveable lower clip, both of which are constructed to inhibit the specimen against slipping or becoming damaged upon tensioning the specimen. More particularly, the specimen is first secured at one end by the upper clip, with the specimen marking near the one end being aligned with the leading edge (e.g., the lowermost edge) of the upper clip so that the specimen hangs freely from the upper clip in a generally relaxed (e.g., unstretched) condition. The weight of the specimen is tared and then the other end of the specimen is secured by the lower clip, with the marking near this end of the specimen being aligned with the leading edge (e.g., uppermost edge) of the lower clip. The lower clip is then moved longitudinally away from the upper clip until the specimen is elongated longitudinally to about 90% of the previously achieved longitudinally elongated configuration (e.g., to a configuration in which the spacing between the markings on the specimen is about 90 mm, which is 90% of 100 mm) of the article. The specimen is maintained in this condition for about thirty seconds. The elastic tension is then measured and recorded using a suitable force gauge. The containment flaps of at least five like articles are tested using the identical procedure and the results are averaged to determine the elastic tension in the flaps.

Although this method has been discussed in terms of containment flaps, a same method can be utilized to determine leg elastic tensions of leg cuffs. In such methods, the upper clamps of the lightbox and the lower clamps holding the weights may be spaced apart to generally coincide with the spacing of the leg cuffs.

As well, if the leg cuffs do not extend for 100 mm in length, the distance chosen may be less, such as 70 mm or the like.

### Thickness Determination Test Method:

To determine a thickness of a sample, and particularly a sample location, a suitable thickness should be used, such as a MITUTOYO 543 Series thickness gauge, available from Mitutoyo-MTI Corporation, Japan. A 1-inch or 3-inch brass foot may be used and the tester should be configured to apply a weight of 0.2 psi. Five samples should be tested, and the results are averaged to provide a thickness value for that sample.

For the articles 10, 210 of the present disclosure, the articles should be tested in an un-folded configuration. Additionally, when testing thickness of a particular location, such as the first location or the second location as described with respect to the Probe Flexibility Test Method, the following instructions should be followed.

Where the location includes particular layers-such as the outer cover, the absorbent system, the bodyside liner, and the fluid management layer, among others, care should be taken to ensure that only such layers are present under the tester's foot. For example, where a chosen location (e.g. such as the first location) includes only the outer cover, the absorbent system, and the bodyside liner, the thickness measurement should be taken such that only these layers are present under the tester's foot. Where the configuration of the article and/or the dimensions of the tester's foot make this impossible, the foot should be positioned to include as to maximize the area under the foot having the same layers as the chosen location. As well, if possible, the areas under the tester's foot having differing layers should be chosen to have fewer layers than the chosen location.

### Bending Stiffness Test Method

Bending stiffness and hysteresis was measured using the KES model FB-2 (available from the Kato Tech Co, Ltd. of Japan). To measure bending, the sample is clamped in an upright position between two chucks. If the sample is greater than 1 mm, then no center adjustment plate is used (the use of and size of any adjustment plate is dependent on the sample thickness and is typically used for samples less than 1 mm in thickness). The samples were cut from products with the center of the sample centered laterally between edges of the absorbent system. The samples should be square and have dimensions of 37.5 mm by 37.5 mm. One of the chucks is stationary while the other rotates in a curvature between 2.5 cm⁻¹ and −2.5 cm⁻¹. The bending tester (KES-FB-2) measures the pure bending properties of a sample for a given range of curvature at a constant rate of 0.5 cm⁻¹/s. The bending curvature 0 to 2.5 cm⁻¹ denotes as the forward bend while bending through curvature 0 to −2.5 cm⁻¹ denotes as the backward bend. The movable chuck moves at a rate of 0.5 cm⁻¹/sec. The amount of moment (grams force*cm/cm) taken to bend the material vs. the curvature is plotted. The samples are oriented within the chuck such that the edge of the sample that was disposed most closely to the product from waist edge is positioned as laterally extending and furthest away from the tester base. For all the materials tested, the following instrument settings were used:

$$Measurement mode = one cycle$$

$$Sensitivity = 2 \times 1$$

$$K Span Control = SET$$

$$Curvature = + / - 2.5 {cm}^{- 1}$$

The KES system algorithm computes the following bending characteristic values:

$$B = bending stiffness ( grams force \times {cm}^{2} / cm )$$

$$2 HB = bending hysteresis ( grams force \times cm / cm )$$

The bending stiffness is defined as the slope of bending moment versus curvature taken between 0.5 cm⁻¹ and 1.5 cm⁻¹, and bending hysteresis is a measure of recovery of the sample after it has been bent and is the distance of the bending and recovery curves at the curvature of 1.0 cm⁻¹. The samples are tested along MD and CD for 5 times each. Higher values bending stiffness indicate higher stiffness of the samples. Higher values of bending hysteresis indicate more difficult for the samples to recover from the bending action.

### EMBODIMENTS

According to a first embodiment, an absorbent article has a central longitudinal axis and has a longitudinal length extending between a front waist edge and a rear waist edge and extends in a lateral direction between longitudinally extending side edges, the absorbent article comprises: a front waist region, a rear waist region, and a crotch region disposed between the front waist region and the rear waist region, a bodyside liner, an outer cover, an absorbent system disposed between the bodyside liner and the outer cover, the absorbent system comprising one or more absorbent layers, a fluid acquisition layer, a first longitudinally extending containment flap comprising a barrier material and one or more longitudinally extending elastic members disposed on a first side of the central longitudinal axis, and a second longitudinally extending containment flap comprising a barrier material and one or more longitudinally extending elastic members disposed on a second side of the central longitudinal axis, each of the pair of longitudinally extending containment flaps having an active region and one or more inactive regions, and leg elastic members disposed outboard of each of the first and second longitudinally extending containment flaps and forming an active leg elastic region between the front waist edge and the rear waist edge, wherein each of the pair of longitudinally extending containment flaps have first inactive regions extending from the front waist edge toward the rear waist edge, the first inactive regions extend for at least 10% of the article longitudinal length, and wherein each of the pair of longitudinally extending containment flaps have an active region disposed between the first inactive regions and the rear waist edge, wherein the absorbent article has an article flexibility of less than or equal to 35 gf at a first location of the article located within the containment flap first inactive regions, and wherein the absorbent article has an article flexibility of greater than or equal to 40 gf at a second location of the article located within the containment flap active region, the article flexibility determined according to the Probe Flexibility Test Method described herein.

In a second embodiment, the absorbent article of embodiment 1 further comprises an absorbent system present at the first location.

In a third embodiment, the absorbent article of any one of embodiments one two further comprises where the fluid acquisition layer is absent at the first location.

In a fourth embodiment, the absorbent article of any one of embodiments one through three further comprises wherein the absorbent system is present at the second location.

In a fifth embodiment, the absorbent article of any one of embodiments one through four further comprises where the fluid acquisition layer is present at the second location.

In a sixth embodiment, the absorbent article of any one of embodiments one through five further comprises wherein the absorbent article has an article flexibility of less than or equal to 27.5 gf at the first location of the article, the article flexibility determined according to the Probe Flexibility Test Method described herein.

In a seventh embodiment, the absorbent article of any one of embodiments one through six further comprises wherein the absorbent article has an article flexibility of less than or equal to 20 gf at the first location of the article, the article flexibility determined according to the Probe Flexibility Test Method described herein.

In an eighth embodiment, the absorbent article of any one of embodiments one through seven further comprises wherein the absorbent article has an article flexibility of greater than or equal to 50 gf at the second location of the article, the article flexibility determined according to the Probe Flexibility Test Method described herein.

In a ninth embodiment, the absorbent article of any one of embodiments one through eight further comprises wherein the absorbent article has an article flexibility of greater than or equal to 60 gf at the second location of the article, the article flexibility determined according to the Probe Flexibility Test Method described herein.

In a tenth embodiment, the absorbent article of any one of embodiments one through nine further comprises wherein the absorbent system comprises a rear region, a middle region, and a front region and further comprises absorbent material, the absorbent material disposed at a basis weight throughout the absorbent system, and wherein an absorbent material basis weight of any one of the rear region, of the middle region, and of the front region does not vary by more than 20% relative to an absorbent material basis weight of any other one of the rear region, the middle region, and the front region.

In an eleventh embodiment, the absorbent article of embodiment 10 further comprises wherein the absorbent material basis weight of any one of the rear region, of the middle region, and of the front region does not vary by more than 10% relative to an absorbent material basis weight of any other one of the rear region, the middle region, and the front region.

In a twelfth embodiment, the absorbent article of any one of embodiments one through eleven further comprises wherein the absorbent article has a first thickness at the first location and a second thickness at the second location, the second thickness being greater than the first thickness by less than 20% of the first thickness, the thicknesses determined according to the Thickness Determination Test Method described herein.

In a thirteenth embodiment, the absorbent article of embodiment twelve further comprises wherein the second thickness is greater than the first thickness by less than 10% of the first thickness, the thicknesses determined according to the Thickness Determination Test Method described herein.

In a fourteenth embodiment, the absorbent article of embodiment twelve further comprises wherein the second thickness is greater than the first thickness by less than 5% of the first thickness, the thicknesses determined according to the Thickness Determination Test Method described herein.

In a fifteenth embodiment, the absorbent article of any one of one of embodiments one through fourteen further comprises wherein the one or more longitudinally extending elastic members of the first longitudinally extending containment flap and the one or more longitudinally extending elastic members of the second longitudinally extending containment flap having flap tensions of between 75 gf and 105 gf, as determined according to the Elastic Member Tension Determination Test Method.

In a sixteenth embodiment, the absorbent article of any one of embodiments one through fifteen further comprises wherein the active leg elastic region extends no closer to the front waist edge than the active regions of each of the pair of longitudinally extending containment flaps.

In a seventeenth embodiment, the absorbent article of any one of embodiments one through sixteen further comprises wherein the active region of each of the pair of longitudinally extending containment flaps extends for between 45-70% of the absorbent article longitudinal length.

In an eighteenth embodiment, the absorbent article of any one of embodiments one through seventeen further comprises wherein the active region of each of the pair of longitudinally extending containment flaps extends for between 45-62.5% of the absorbent article longitudinal length.

In a nineteenth embodiment, the absorbent article of any one of embodiments one through eighteen further comprises wherein the active region of each of the pair of longitudinally extending containment flaps extends for between 45-55% of the absorbent article longitudinal length.

In a twentieth embodiment, the absorbent article of any one of embodiments one through nineteen further comprises wherein the absorbent article has a thickness at the second location of less than 4 mm, the thicknesses determined according to the Thickness Determination Test Method described herein.

In a twenty-first embodiment, the absorbent article of any one of embodiments one through twenty further comprises wherein the absorbent article has a thickness at the second location of less than 3 mm, the thicknesses determined according to the Thickness Determination Test Method described herein.

In a twenty-second embodiment, the absorbent article of any one of embodiments one through twenty-one further comprises wherein the absorbent article has a graphic element disposed on the outer cover, and wherein the graphic element extends continuously between the one or more inactive zones and the active zone of each of the pair of longitudinally extending containment flaps.

In a twenty-third embodiment, the absorbent article of embodiment twenty-two further comprises wherein the graphic element is an anthropomorphous graphic element.

In a twenty-fourth embodiment, an absorbent article has a central longitudinal axis and has a longitudinal length extending between a front waist edge and a rear waist edge and extends in a lateral direction between longitudinally extending side edges, the absorbent article comprises a front waist region, a rear waist region, and a crotch region disposed between the front waist region and the rear waist region, a bodyside liner, an outer cover, an absorbent system disposed between the bodyside liner and the outer cover, the absorbent system comprising one or more absorbent layers and further comprising a rear region, a middle region, and a front region, the absorbent system comprised of absorbent material with the absorbent material disposed at a basis weight throughout the absorbent system, wherein an absorbent material basis weight of any one of the rear region, of the middle region, and of the front region does not vary by more than 20% relative to an absorbent material basis weight of any other one of the rear region, the middle region, and the front region, a fluid acquisition layer extending between a front edge disposed proximate the article front waist edge and a rear edge disposed proximate the article rear waist edge, a first longitudinally extending containment flap comprising a barrier material and one or more longitudinally extending elastic members disposed on a first side of the central longitudinal axis, and a second longitudinally extending containment flap comprising a barrier material and one or more longitudinally extending elastic members disposed on a second side of the central longitudinal axis, each of the pair of longitudinally extending containment flaps having an active region and one or more inactive regions, and leg elastic members disposed outboard of each of the first and second longitudinally extending containment flaps and forming active leg elastic region between the front waist edge and the rear waist edge, wherein each of the pair of longitudinally extending containment flaps have first inactive regions extending from the front waist edge toward the rear waist edge, the first inactive regions extend for at least 10% of the article longitudinal length, and wherein each of the pair of longitudinally extending containment flaps have active regions disposed between the first inactive regions and the rear waist edge, wherein the fluid acquisition layer front edge is disposed no further away from the absorbent article front waist edge than the active regions of each of the pair of longitudinally extending containment flaps, and wherein the absorbent article has an article flexibility of less than or equal to 40 gf at a first location of the article located within the first inactive regions and where the absorbent system is present, the article flexibility determined according to the Probe Flexibility Test Method described herein.

In a twenty-fifth embodiment, the absorbent article of embodiment twenty-four further comprises wherein the absorbent article has a thickness at a second location disposed within the active regions of the pair of longitudinally extending containment flaps and where the absorbent system and the fluid acquisition layer are present, the thickness being less than 4 mm, the thicknesses determined according to the Thickness Determination Test Method described herein.

In a twenty-sixth embodiment, the absorbent article of any one of embodiment twenty-four and embodiment twenty-five further comprises wherein the absorbent article has a thickness at a second location disposed within the active regions of the pair of longitudinally extending containment flaps and where the absorbent system and the fluid acquisition layer are present, the thickness being less than 3 mm, the thicknesses determined according to the Thickness Determination Test Method described herein.

In a twenty-seventh embodiment, the absorbent article of any one of embodiments twenty-four through twenty-six further comprises wherein the absorbent article has a first thickness at the first location, a second thickness at a second location disposed within the active regions of the pair of longitudinally extending containment flaps and where the absorbent system and the fluid acquisition layer are present, and wherein the second thickness is greater than the first thickness by less than 20% of the first thickness, the thicknesses determined according to the Thickness Determination Test Method described herein.

In a twenty-eighth embodiment, the absorbent article of embodiment twenty-seven further comprises wherein the second thickness is greater than the first thickness by less than 10% of the first thickness, the thicknesses determined according to the Thickness Determination Test Method described herein.

In a twenty-ninth embodiment, the absorbent article of any one of embodiment twenty-seven and embodiment twenty-eight further comprises wherein the second thickness is greater than the first thickness by less than 5% of the first thickness, the thicknesses determined according to the Thickness Determination Test Method described herein.

In a thirtieth embodiment, the absorbent article of any one of embodiments twenty-four through twenty-nine further comprises wherein the absorbent material basis weight of any one of the rear region, of the middle region, and of the front region does not vary by more than 10% relative to an absorbent material basis weight of any other one of the rear region, the middle region, and the front region.

In a thirty-first embodiment, the absorbent article of any one of embodiments twenty-four through thirty further comprises wherein the one or more longitudinally extending elastic members of the first longitudinally extending containment flap and the one or more longitudinally extending elastic members of the second longitudinally extending containment flap having flap tensions of between 75 gf and 105 gf, as determined according to the Elastic Member Tension Determination Test Method.

In a thirty-second embodiment, the absorbent article of any one of embodiments twenty-four through thirty-one further comprises wherein the active region of each of the pair of longitudinally extending containment flaps extends for between 45-70% of the absorbent article longitudinal length.

In a thirty-third embodiment, the absorbent article of any one of embodiments twenty-four through thirty-two further comprises wherein the active region of each of the pair of longitudinally extending containment flaps extends for between 45-62.5% of the absorbent article longitudinal length.

In a thirty-fourth embodiment, the absorbent article of one of embodiments twenty-four through thirty-three further comprises wherein the active region of each of the pair of longitudinally extending containment flaps extends for between 45-55% of the absorbent article longitudinal length.

In a thirty-fifth embodiment, the absorbent article of any one of embodiments twenty-four through thirty-four further comprises wherein the active leg elastic region begins no closer to the front waist edge than the active regions of each of the pair of longitudinally extending containment flaps.

In a thirty-sixth embodiment, the absorbent article of any one of embodiments twenty-four through thirty-five further comprises wherein the absorbent article has a graphic element disposed on the outer cover, and wherein the graphic element extends continuously between the one or more inactive zones and the active zone of each of the pair of longitudinally extending containment flaps.

In a thirty-seventh embodiment, the absorbent article of embodiment thirty-six further comprises wherein the graphic element is an anthropomorphous graphic element.

In a thirty-eighth embodiment, an absorbent article has a central longitudinal axis and has a longitudinal length extending between a front waist edge and a rear waist edge and extends in a lateral direction between longitudinally extending side edges, the absorbent article comprises a front waist region, a rear waist region, and a crotch region disposed between the front waist region and the rear waist region, a bodyside liner, an outer cover, an absorbent system extending between an absorbent system front edge disposed proximate the article front waist edge and an absorbent system rear edge disposed proximate the article rear waist edge and disposed between the bodyside liner and the outer cover, the absorbent system comprising one or more absorbent layers, a fluid acquisition layer extending between a fluid acquisition layer front edge disposed proximate the article front waist edge and a fluid acquisition layer rear edge disposed proximate the article rear waist edge, wherein the fluid acquisition layer front edge is disposed closer to the article rear waist edge than the absorbent system front edge, a first longitudinally extending containment flap comprising a barrier material and one or more longitudinally extending elastic members disposed on a first side of the central longitudinal axis, and a second longitudinally extending containment flap comprising a barrier material and one or more longitudinally extending elastic members disposed on a second side of the central longitudinal axis, each of the pair of longitudinally extending containment flaps having an active region and one or more inactive regions, and leg elastic members disposed outboard of each of the first and second longitudinally extending containment flaps and forming active leg elastic region between the front waist edge and the rear waist edge, wherein each of the pair of longitudinally extending containment flaps have first inactive regions extending from the front waist edge toward the rear waist edge, the first inactive regions extend for at least 10% of the article longitudinal length, and wherein each of the pair of longitudinally extending containment flaps have active regions disposed between the first inactive regions and the rear waist edge, wherein the fluid acquisition layer front edge is disposed no further away from the absorbent article front waist edge than the active regions of each of the pair of longitudinally extending containment flaps, and wherein the absorbent article has an article flexibility of less than or equal to 40 gf at a first location of the article located within the first inactive regions and where the absorbent system is present, the article flexibility determined according to the Probe Flexibility Test Method described herein.

In a thirty-ninth embodiment, the absorbent article of embodiment thirty-eight further comprises where the fluid acquisition layer is absent at the first location.

In a fortieth embodiment, the absorbent article of any one of embodiment thirty-eight and embodiment thirty-nine further comprises wherein the absorbent article has an article flexibility of less than or equal to 27.5 gf at the first location of the article, the article flexibility determined according to the Probe Flexibility Test Method described herein.

In a forty-first embodiment, the absorbent article of any one of embodiments thirty-eight through forty further comprises wherein the absorbent article has a thickness at a second location disposed within the active regions of the pair of longitudinally extending containment flaps and where the absorbent system and the fluid acquisition layer are present, the thickness being less than 4 mm, the thicknesses determined according to the Thickness Determination Test Method described herein.

In a forty-second embodiment, the absorbent article of any one of embodiments thirty-eight through forty-one further comprises wherein the absorbent article has a thickness at a second location disposed within the active regions of the pair of longitudinally extending containment flaps and where the absorbent system and the fluid acquisition layer are present, the thickness being less than 3 mm, the thicknesses determined according to the Thickness Determination Test Method described herein.

In a forty-third embodiment, the absorbent article of any one of embodiments thirty-eight through forty-two further comprises wherein the absorbent article has a first thickness at the first location, a second thickness at a second location disposed within the active regions of the pair of longitudinally extending containment flaps and where the absorbent system and the fluid acquisition layer are present, and wherein the second thickness is greater than the first thickness by less than 20% of the first thickness, the thicknesses determined according to the Thickness Determination Test Method described herein.

In a forty-fourth embodiment, the absorbent article of embodiment forty-three further comprises wherein the second thickness is greater than the first thickness by less than 10% of the first thickness, the thicknesses determined according to the Thickness Determination Test Method described herein.

In a forty-fifth embodiment, the absorbent article of embodiment forty-three further comprises wherein the second thickness is greater than the first thickness by less than 5% of the first thickness, the thicknesses determined according to the Thickness Determination Test Method described herein.

In a forty-sixth embodiment, the absorbent article of any one of embodiments thirty-eight through forty-five further comprises wherein the active region of each of the pair of longitudinally extending containment flaps extends for between 45-70% of the absorbent article longitudinal length.

In a forty-seventh embodiment, the absorbent article of any one of embodiments thirty-eight through forty-six further comprises wherein the active region of each of the pair of longitudinally extending containment flaps extends for between 45-62.5% of the absorbent article longitudinal length.

In a forty-eighth embodiment, the absorbent article of any one of embodiments thirty-eight through forty-seven further comprises wherein the active region of each of the pair of longitudinally extending containment flaps extends for between 45-55% of the absorbent article longitudinal length.

In a forty-ninth embodiment, the absorbent article of any one of embodiments thirty-eight through forty-eight further comprises wherein the active leg elastic region begins no closer to the front waist edge than the active regions of each of the pair of longitudinally extending containment flaps.

In a fiftieth embodiment, the absorbent article of any one of embodiments thirty-eight through forty-nine further comprises wherein the one or more longitudinally extending elastic members of the first longitudinally extending containment flap and the one or more longitudinally extending elastic members of the second longitudinally extending containment flap having flap tensions of between 75 gf and 105 gf, as determined according to the Elastic Member Tension Determination Test Method.

In a fifty-first embodiment, the absorbent article of any one of embodiments thirty-eight through fifty further comprises wherein the active leg elastic region begins no closer to the front waist edge than the active regions of each of the pair of longitudinally extending containment flaps.

In a fifty-second embodiment, the absorbent article of any one of embodiments thirty-eight through fifty-one further comprises wherein the absorbent article has a graphic element disposed on the outer cover, and wherein the graphic element extends continuously between the one or more inactive zones and the active zone of each of the pair of longitudinally extending containment flaps.

In a fifty-third embodiment, the absorbent article of embodiment fifty-two further comprises wherein the graphic element is an anthropomorphous graphic element.

In a fifty-fourth embodiment, the absorbent article of any one of embodiments one through fifty-three further comprises wherein the absorbent article has a probe flexibility at the first location, as measured according to the Probe Flexibility Test Method, and a thickness, as determined according to the Thickness Determination Test Method, such that the probe flexibility multiplied by a cube of the thickness is greater than or equal to 2534.

What is claimed is:

1. An absorbent article having a central longitudinal axis and having a longitudinal length extending between a front waist edge and a rear waist edge and extends in a lateral direction between longitudinally extending side edges, the absorbent article comprising:
a front waist region, a rear waist region, and a crotch region disposed between the front waist region and the rear waist region;
a bodyside liner;
an outer cover;
an absorbent system disposed between the bodyside liner and the outer cover, the absorbent system comprising one or more absorbent layers;
a fluid acquisition layer;
a first longitudinally extending containment flap comprising a barrier material and one or more longitudinally extending elastic members disposed on a first side of the central longitudinal axis, and a second longitudinally extending containment flap comprising a barrier material and one or more longitudinally extending elastic members disposed on a second side of the central longitudinal axis, each of the pair of longitudinally extending containment flaps having an active region and one or more inactive regions; and
leg elastic members disposed outboard of each of the first and second longitudinally extending containment flaps and forming an active leg elastic region between the front waist edge and the rear waist edge;
wherein each of the pair of longitudinally extending containment flaps have first inactive regions extending from the front waist edge toward the rear waist edge, the first inactive regions extend for at least 10% of the article longitudinal length, and wherein each of the pair of longitudinally extending containment flaps have an active region disposed between the first inactive regions and the rear waist edge;
wherein the absorbent article has an article flexibility of less than or equal to 35 gf at a first location of the article located within the containment flap first inactive regions, and wherein the absorbent article has an article flexibility of greater than or equal to 40 gf at a second location of the article located within the containment flap active region, the article flexibility determined according to the Probe Flexibility Test Method described herein.

2. The absorbent article of claim 1, wherein the absorbent system is present at the first location.

3. The absorbent article of claim 1, where the fluid acquisition layer is absent at the first location.

4. The absorbent article of claim 1, wherein the absorbent system is present at the second location.

5. The absorbent article of claim 1, where the fluid acquisition layer is present at the second location.

6. The absorbent article of claim 1, wherein the absorbent article has an article flexibility of less than or equal to 27.5 gf at the first location of the article, the article flexibility determined according to the Probe Flexibility Test Method described herein.

7. The absorbent article of claim 1, wherein the absorbent article has an article flexibility of greater than or equal to 50 gf at the second location of the article, the article flexibility determined according to the Probe Flexibility Test Method described herein.

8. The absorbent article of claim 1, wherein the absorbent system comprises a rear region, a middle region, and a front region and further comprises absorbent material, the absorbent material disposed at a basis weight throughout the absorbent system, and wherein an absorbent material basis weight of any one of the rear region, of the middle region, and of the front region does not vary by more than 20% relative to an absorbent material basis weight of any other one of the rear region, the middle region, and the front region.

9. The absorbent article of claim 1, wherein the absorbent article has a first thickness at the first location and a second thickness at the second location, the second thickness being greater than the first thickness by less than 20% of the first thickness, the thicknesses determined according to the Thickness Determination Test Method described herein.

10. The absorbent article of claim 1, wherein the absorbent article has a thickness at the second location of less than 4 mm, the thicknesses determined according to the Thickness Determination Test Method described herein.

11. The absorbent article of claim 1, wherein the absorbent article has a graphic element disposed on the outer cover, and wherein the graphic element extends continuously between the one or more inactive zones and the active zone of each of the pair of longitudinally extending containment flaps.

12. The absorbent article of claim 11, wherein the graphic element is an anthropomorphous graphic element.

13. An absorbent article having a central longitudinal axis and having a longitudinal length extending between a front waist edge and a rear waist edge and extends in a lateral direction between longitudinally extending side edges, the absorbent article comprising:
a front waist region, a rear waist region, and a crotch region disposed between the front waist region and the rear waist region;
a bodyside liner;
an outer cover;
an absorbent system disposed between the bodyside liner and the outer cover, the absorbent system comprising one or more absorbent layers and further comprising a rear region, a middle region, and a front region, the absorbent system comprised of absorbent material with the absorbent material disposed at a basis weight throughout the absorbent system, wherein an absorbent material basis weight of any one of the rear region, of the middle region, and of the front region does not vary by more than 20% relative to an absorbent material basis weight of any other one of the rear region, the middle region, and the front region;
a fluid acquisition layer extending between a front edge disposed proximate the article front waist edge and a rear edge disposed proximate the article rear waist edge;
a first longitudinally extending containment flap comprising a barrier material and one or more longitudinally extending elastic members disposed on a first side of the central longitudinal axis, and a second longitudinally extending containment flap comprising a barrier material and one or more longitudinally extending elastic members disposed on a second side of the central longitudinal axis, each of the pair of longitudinally extending containment flaps having an active region and one or more inactive regions; and
leg elastic members disposed outboard of each of the first and second longitudinally extending containment flaps and forming active leg elastic region between the front waist edge and the rear waist edge;
wherein each of the pair of longitudinally extending containment flaps have first inactive regions extending from the front waist edge toward the rear waist edge, the first inactive regions extend for at least 10% of the article longitudinal length, and wherein each of the pair of longitudinally extending containment flaps have active regions disposed between the first inactive regions and the rear waist edge;
wherein the fluid acquisition layer front edge is disposed no further away from the absorbent article front waist edge than the active regions of each of the pair of longitudinally extending containment flaps; and
wherein the absorbent article has an article flexibility of less than or equal to 40 gf at a first location of the article located within the first inactive regions and where the absorbent system is present, the article flexibility determined according to the Probe Flexibility Test Method described herein.

14. The absorbent article of claim 13, wherein the absorbent article has a thickness at a second location disposed within the active regions of the pair of longitudinally extending containment flaps and where the absorbent system and the fluid acquisition layer are present, the thickness being less than 4 mm, the thicknesses determined according to the Thickness Determination Test Method described herein.

15. The absorbent article of claim 13, wherein the absorbent article has a first thickness at the first location, a second thickness at a second location disposed within the active regions of the pair of longitudinally extending containment flaps and where the absorbent system and the fluid acquisition layer are present, and wherein the second thickness is greater than the first thickness by less than 20% of the first thickness, the thicknesses determined according to the Thickness Determination Test Method described herein.

16. The absorbent article of claim 13, wherein the one or more longitudinally extending elastic members of the first longitudinally extending containment flap and the one or more longitudinally extending elastic members of the second longitudinally extending containment flap having flap tensions of between 75 gf and 105 gf, as determined according to the Elastic Member Tension Determination Test Method.

17. An absorbent article having a central longitudinal axis and having a longitudinal length extending between a front waist edge and a rear waist edge and extends in a lateral direction between longitudinally extending side edges, the absorbent article comprising:
a front waist region, a rear waist region, and a crotch region disposed between the front waist region and the rear waist region;
a bodyside liner;
an outer cover;
an absorbent system extending between an absorbent system front edge disposed proximate the article front waist edge and an absorbent system rear edge disposed proximate the article rear waist edge and disposed between the bodyside liner and the outer cover, the absorbent system comprising one or more absorbent layers;
a fluid acquisition layer extending between a fluid acquisition layer front edge disposed proximate the article front waist edge and a fluid acquisition layer rear edge disposed proximate the article rear waist edge, wherein the fluid acquisition layer front edge is disposed closer to the article rear waist edge than the absorbent system front edge;
a first longitudinally extending containment flap comprising a barrier material and one or more longitudinally extending elastic members disposed on a first side of the central longitudinal axis, and a second longitudinally extending containment flap comprising a barrier material and one or more longitudinally extending elastic members disposed on a second side of the central longitudinal axis, each of the pair of longitudinally extending containment flaps having an active region and one or more inactive regions; and
leg elastic members disposed outboard of each of the first and second longitudinally extending containment flaps and forming active leg elastic region between the front waist edge and the rear waist edge;
wherein each of the pair of longitudinally extending containment flaps have first inactive regions extending from the front waist edge toward the rear waist edge, the first inactive regions extend for at least 10% of the article longitudinal length, and wherein each of the pair of longitudinally extending containment flaps have active regions disposed between the first inactive regions and the rear waist edge;
wherein the fluid acquisition layer front edge is disposed no further away from the absorbent article front waist edge than the active regions of each of the pair of longitudinally extending containment flaps; and
wherein the absorbent article has an article flexibility of less than or equal to 40 gf at a first location of the article located within the first inactive regions and where the absorbent system is present, the article flexibility determined according to the Probe Flexibility Test Method described herein.

18. The absorbent article of claim 17, where the fluid acquisition layer is absent at the first location.

19. The absorbent article of claim 17, wherein the active leg elastic region begins no closer to the front waist edge than the active regions of each of the pair of longitudinally extending containment flaps.

20. The absorbent article of claim 17, wherein the absorbent article has a probe flexibility at the first location, as measured according to the Probe Flexibility Test Method, and a thickness, as determined according to the Thickness Determination Test Method, such that the probe flexibility multiplied by a cube of the thickness is greater than or equal to 2534.
